(19) United States
(12) Patent Application Publication
(10) Pub. No.: US 2024/0050436 A1
(43) Pub. Date: Feb. 15, 2024

(54) COMPOSITIONS AND METHODS USED FOR RETINAL ISCHEMIA OR DIABETES

(71) Applicants: The United States GOVERNMENT As Represented By The Department of Veterans Affairs, Washington, DC (US); THE BOARD OF TRUSTEES OF THE LELAND STANFORD JUNIOR UNIVERSITY, STANFORD, CA (US)

(72) Inventors: AZZA EL-REMESSY, Martinez, GA (US); FRANK LONGO, Menlo Park, CA (US)

(21) Appl. No.: 18/491,249

(22) Filed: Oct. 20, 2023

Related U.S. Application Data
(63) Continuation of application No. 18/060,160, filed on Nov. 30, 2022, now abandoned.
(60) Provisional application No. 63/284,489, filed on Nov. 30, 2021.

(51) Int. Cl. A61K 31/5375; A61P 27/02; A61K 38/18; A61K 35/28; A61P 13/02; A61P 13/12

(52) U.S. Cl. CPC A61K 31/5375; A61P 27/02; A61K 38/1866; A61K 35/28; A61P 13/02; A61P 13/12

(57) ABSTRACT

Disclosed are methods of treating an ocular disease associated with retinal ischemic injury in a subject comprising administering to the subject a p75^{NTR} modulator, a MSC comprising decreased p75^{NTR} expression or activity or a MSC secretome collected from a MSC comprising decreased p75^{NTR} expression or activity. Disclosed are methods of treating late stage diabetes in a subject comprising administering to the subject in need thereof a p75^{NTR} modulator, a MSC comprising decreased p75^{NTR} expression or activity or a MSC secretome collected from a MSC comprising decreased p75^{NTR} expression or activity.

### CROSS-REFERENCE TO RELATED APPLICATIONS

This application is a Continuation of U.S. Non-Provisional patent application Ser. No. 18/060,160, filed on Nov. 30, 2022, which claims the benefit of U.S. Provisional Patent Application No. 63/284,489, filed on Nov. 30, 2021, each of which is incorporated by reference herein in its entirety.

### STATEMENT REGARDING FEDERALLY SPONSORED RESEARCH

This invention was made with government support under Grant Number R01-EY-022408 awarded by the National Institutes of Health. The government has certain rights in this invention.

### REFERENCE TO SEQUENCE LISTING

The Sequence Listing submitted Oct. 20, 2023 as an xml file named “37759_0397_U3_Sequence_Listing,” created on Oct. 19, 2023, and having a size of 15,871 bytes is hereby incorporated by reference pursuant to 37 C.F.R. § 1.52(e)(5).

### BACKGROUND

Vision loss associated with ischemic diseases, such as retinopathy of prematurity, diabetic retinopathy and optic neuropathy, are often due to the growth of malfunctional retinal capillaries, in response to retinal ischemia. Significant progress has been made in combating abnormal vascular proliferation, including laser photocoagulation, anti-angiogenic vascular endothelial growth factor (VEGF) inhibitors and angiostatic steroids. Nevertheless, these therapeutic approaches fail to treat ischemic areas with a large degree of tissue injury in addition to local and systemic complications associated with their use. Thus, there is an urgent need to develop new therapeutic alternatives to combat early retinal ischemia and thus diminish subsequent destructive neovascularization.

The p75NTR, a common receptor for all neurotrophins along with their precursor forms, can exert multiple functions, including cell survival, death or angiogenesis, according to cell context. Among increasing prospective stem cells markers, the p75NTR receptor, also known as CD271, enriches several progenitor/stem cell subtypes. P75NTR was first identified as a genuine neural crest stem cell marker. Since then, it has been widely used to isolate putative stem cells from neural crest-derived tissues. The p75NTR was shown to directly inhibit the differentiation of MSCS into multiple cell types. Several studies reported a causal relationship of p75NTR in mediating retinal inflammation, barrier dysfunction and development of acellular capillaries. Nonetheless, p75NTR expression and function in MSCs biology, as well as its underlying mechanisms, have not been fully studied. In the current study, the impact of modulating p75NTR expression or activity on the surface of mesenchymal stem cells (MSCs) in increasing their vascular homing and repair in ischemic retina using an ischemia/reperfusion (I/R) mouse model was evaluated. Further, the protective action of pharmacological inhibition of p75NTR on improving visual acuity post I/R injury was investigated.

### BRIEF SUMMARY

Disclosed are methods of treating an ocular disease associated with retinal ischemic injury in a subject comprising administering to the subject in need thereof a p75^{NTR} modulator.

Disclosed are methods of treating an ocular disease associated with retinal ischemic injury in a subject comprising administering to the subject in need thereof a mesenchymal stem cell (MSC) comprising decreased p75^{NTR} expression or activity.

Disclosed are methods of treating an ocular disease associated with retinal ischemic injury in a subject comprising administering to the subject in need thereof a mesenchymal stem cell (MSC) secretome collected from a MSC comprising decreased p75^{NTR} expression or activity.

Disclosed are methods of improving visual acuity in a subject having a retinal ischemic injury comprising administering to the subject in need thereof a p75^{NTR} modulator.

Disclosed are methods of improving visual acuity in a subject having a retinal ischemic injury comprising administering to the subject in need thereof a mesenchymal stem cell (MSC) comprising decreased p75^{NTR} expression or activity.

Disclosed are methods of improving visual acuity in a subject having a retinal ischemic injury comprising administering to the subject a mesenchymal stem cell (MSC) secretome collected from one or more MSCs comprising decreased p75^{NTR} expression or activity.

Disclosed are methods of preventing ischemia-induced retinal capillary degeneration in a subject comprising administering to the subject in need thereof a p75^{NTR} modulator, wherein the p75^{NTR} modulator inhibits the expression or activity of p75^{NTR} in one or more MSCs in the retina of the subject.

Disclosed are methods of preventing ischemia-induced retinal capillary degeneration in a subject comprising administering to the subject one or more MSCs comprising decreased p75^{NTR} expression or activity.

Disclosed are methods preventing ischemia-induced retinal capillary degeneration in a subject comprising administering to the subject a MSC secretome collected from one or more MSCs comprising decreased p75^{NTR} expression or activity.

Disclosed are methods of increasing vascular protection in an ischemic retina of a subject comprising administering to the retina of the subject in need thereof a p75^{NTR} modulator, wherein the p75^{NTR} modulator inhibits expression or activity of p75^{NTR} in one or more MSCs in the retina of the subject.

Disclosed are methods of increasing vascular protection in an ischemic retina of a subject comprising administering to the retina of the subject one or more MSCs comprising decreased p75^{NTR} expression or activity.

Disclosed are methods of increasing vascular protection in an ischemic retina of a subject comprising administering to the retina of the subject a MSC secretome collected from one or more MSCs comprising decreased p75^{NTR} expression or activity.

Disclosed are methods of increasing vascular homing of one or more MSCs at a site of ischemic injury in a subject comprising administering to the subject in need thereof a p75^{NTR} modulator, wherein the p75^{NTR} modulator inhibits expression or activity of p75^{NTR} in the one or more MSCs.

Disclosed are methods of increasing vascular homing of one or more MSCs at a site of ischemic injury in a subject comprising administering to the subject one or more MSCs comprising decreased p75^{NTR} expression or activity.

Disclosed are methods of increasing vascular homing of one or more MSCs at a site of ischemic injury in a subject comprising administering to the subject a MSC secretome collected from one or more MSCs comprising decreased p75^{NTR} expression or activity.

Disclosed are methods of altering a MSC secretome comprising inhibiting the expression or activity of p75^{NTR} in the MSC. In some aspects, altering a MSC secretome can be improving a MSC's secretome.

Disclosed are methods of increasing survival factors in a HRE comprising contacting the HRE with one or more MSCs having decreased p75^{NTR} expression or activity.

Disclosed are methods of increasing angiogenic effects of a HRE comprising contacting the HRE with one or more MSCs having decreased p75^{NTR} expression or activity, wherein HRE migration is increased.

Disclosed are methods of decreasing urinary albumin excretion rate in a subject having diabetes comprising administering to the subject a p75^{NTR} modulator.

Disclosed are methods of decreasing inflammation in the kidney of a subject having diabetes comprising administering to the subject a p75^{NTR} modulator.

Disclosed are methods of decreasing IL-1β in a subject having diabetes comprising administering to the subject a p75^{NTR} modulator, wherein IL-1β is decreased in one or both kidneys after administration of the p75^{NTR} modulator.

Disclosed are methods of decreasing IL-6 in a subject having diabetes comprising administering to the subject in need thereof a p75^{NTR} modulator, wherein IL-6 is decreased in one or both kidneys after administration of the p75^{NTR} modulator.

Disclosed are methods of decreasing TGF-β in a subject having diabetes comprising administering to the subject in need thereof a p75^{NTR} modulator, wherein TGF-β is decreased in one or both kidneys after administration of the p75^{NTR} modulator.

Disclosed are methods of decreasing one or more markers of fibrosis in a subject having diabetes comprising administering to the subject in need thereof a p75NTR modulator, wherein one or more markers of fibrosis is decreased in one or both kidneys after administration of the p75NTR modulator.

Disclosed are methods of decreasing fibrotic tissue in a kidney of a subject having diabetes comprising administering to the subject in need thereof a p75^{NTR} modulator.

Disclosed are methods of treating late stage diabetes in a subject comprising administering to the subject in need thereof a p75^{NTR} modulator.

Additional advantages of the disclosed method and compositions will be set forth in part in the description which follows, and in part will be understood from the description, or may be learned by practice of the disclosed method and compositions. The advantages of the disclosed method and compositions will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

The accompanying drawings, which are incorporated in and constitute a part of this specification, illustrate several embodiments of the disclosed method and compositions and together with the description, serve to explain the principles of the disclosed method and compositions.

FIG. 1A, FIG. 1B, and FIG. 1C show deletion of p75^{NTR} reduced the development of acellular capillaries in ischemic retinas. (FIG. 1A, FIG. 1B) Representative trypsin-digested and PASH-stained retinas of WT and p75^{NTR-/-} mice subjected to I/R. Bright field imaging showed an increased number of acellular capillaries (red arrows) in WT that was attenuated in p75^{NTR-/-} mice (20× magnification. (FIG. 1C) Statistical analysis using two-way ANOVA showed significant impact of disease condition (ischemia) and gene deletion on the number of acellular capillaries. I/R significantly increased the number of acellular capillaries in WT retinas when compared to shams. This effect was ameliorated in retinas of p75^{NTR-/-} mice (*, significant using two-way ANOVA, n=4-5).

FIGS. 2A-2D show MSCs incorporate to ischemic retinal vasculature and decreased acellular capillaries in WT and p75^{NTR-/-} ischemic retinas. (FIG. 2A, FIG. 2B) Confocal images of retinal flat mounts stained with isolectin (red), showing incorporation of intravitreally-injected GFP-labeled MSCs (green) into WT retinas subjected to I/R (40× magnification). (FIG. 2A) Different regions of flat-mounted retina 3 days post injections showing some MSCs morphed into amoboid/satellite-like shape (green, left panel) and some MSCs picked up isolectin stain (yellow, right panel). (FIG. 2B) Different regions of flat-mounted retina 1 week post injection showing, in the left panel, that some of the MSCs appeared perivascular (green) wrapped around retinal capillaries (red) and some other MSCs (right panel) maintained satellite-like shape and co-stained with isolectin (yellow) within retinal capillaries (red), indicating integration into retinal vasculature. (FIG. 2C) Representative trypsin-digested and PASH-stained retinas of WT and p75^{NTR-/-} mice subjected to I/R and receiving GFP-labeled MSCs. Bright field imaging showed decreased count for acellular capillaries (red arrows, 20× magnification). (FIG. 2D) Bar graph and statistical analysis for acellular capillaries count in WT and p75^{NTR-/-} retinas subjected to I/R and receiving GFP-labeled MSCs. Ischemic WT retinas still showed a significant increase in the count of acellular capillaries that was completely ameliorated in p75^{NTR-/-} retinas following MSCs injection (*, significant using two-way ANOVA, n=7-11).

FIGS. 3A-3C show silencing p75^{NTR} expression in MSCs increased vascular homing and protection in ischemic retinas. (FIG. 3A) Confocal images of ischemic WT retinal flat mounts stained with Isolectin-GS (red), showing numerous GFP-labeled MSCs (green) recruited to retinal capillaries (red) after silencing p75^{NTR} expression when compared to scrambled-MSCs (40× magnification). (FIG. 3B) Representative trypsin-digested and PASH-stained ischemic WT retinas showing decreased acellular capillaries (red arrows) in ischemic retinas receiving MSCs that do not express p75^{NTR} (20× magnification). (FIG. 3C) Bar graph and statistical analysis for acellular capillaries count in ischemic WT retinas receiving Scrambled mRNA-treated or siRNA-treated MSCs against p75^{NTR}. Number of acellular capillaries was significantly higher in retinas receiving p75^{NTR}-expressing MSCs but not p75^{NTR}-knocked down cells (*, significant using two-way ANOVA, n=4).

FIGS. 4A-4D show silencing p75^{NTR} expression improves MSCs-secretome. Representative micrograph of Western blotting (FIG. 4A) and statistical analysis (FIG. 4B, FIG. 4C and FIG. 4D) of GFP-MSCs CM showed 1.8-Fold increase in SDF-1-a, 6-Fold increase in VEGF-A and 5.8-Fold increase in NGF, upon silencing the expression of p75^{NTR} (* p<0.05, n=3. Significance detected by unpaired student T-test).

FIGS. 5A-5F show silencing p75^{NTR} expression on MSCs improves paracrine effect in HREs. RT-PCR analysis showed that treatment of HREs with CM of p75^{NTR}-Silenced MSCs significantly increased gene expression of angiogenic and survival factor transcripts, including VEGF-A (FIG. 5A), Akt (FIG. 5B), Bcl-2 (FIG. 5C) and some of the apoptotic markers including BAX (FIG. 5D), but no change was observed in p53 (FIG. 5E) or caspase-3 (FIG. 5F) transcripts when compared to HREs treated with CM of control cells. *, significant at p<0.05; NS, not significant. Significance was detected by unpaired student T-test, n=3.

FIGS. 6A-6E show silencing p75^{NTR} expression on MSCs improves angiogenic response in HREs. (FIG. 6A and FIG. 6B) Representative images and statistical analysis of HREs migration shows significant increase in HREs migration (distance marked from original plating line marked with blue pen) after 12 h treatment with CM of p75^{NTR}-silenced MSCs (*, significance using one-way ANOVA test, n=5-7. (FIG. 6C-FIG. 6E) Representative phase contrast images (FIG. 6C) and statistical analysis for tube length (FIG. 6D) and number of tube junction (FIG. 6E) formed in vitro by HREs after 24 h treatment with conditioned medium of p75^{NTR}-silenced MSCs. Results showed significant increase in tube length and number of tube junctions (*, significance using one-way ANOVA test, n=3).

FIGS. 7A-7I show modulating p75^{NTR} on MSCs using LM11A-31 improved their secretome and improved paracrine effect in HREs. Representative Western blotting (FIG. 7A) and statistical analysis of MSCs secretome treated with the pharmacologic modulator of p75^{NTR}; LM11A-31. (FIG. 7B-FIG. 7D) showing ˜14.9-Fold increase in SDF-1a (FIG. 7B), 1.6-Fold increase in VEGF-A (FIG. 7C) and ˜4-Fold increase in NGF (FIG. 7D). * p<0.05, Significance was detected by unpaired Student t-test, n=3. RT-PCR analysis showed that treatment of HREs with CM of LM11A-31-treated MSCs significantly increased gene expression of the survival factor transcripts VEGF-A (FIG. 7E), Akt (FIG. 7F), Bcl-2 (FIG. 7G) and some of the apoptotic markers, such as Bax (H), but not p53 (I). *, Significant at p<0.05; NS, not significant. Significance was detected by unpaired Student t-test, n=3.

FIGS. 8A-8E show conditioned medium of LM11A-31-treated MSCs improved angiogenic response in HREs. (FIG. 8A and FIG. 8B) Representative micrographs and statistical analysis of HREs migration shows a significant increase in HREs migration (distance marked from original plating line marked with blue pen) after 12 h treatment with CM of LM11A-31-modulated MSCs (*, significance using unpaired student t-test, n=5-7). (FIG. 8C-FIG. 8E) Representative phase contrast images (FIG. 8C) and statistical analysis for tube length (FIG. 8D) and number of tube junction (FIG. 8E) formed in vitro by HREs after 24 h treatment with CM of LM11A-31-modulated MSCs. Results showed a significant increase in tube length and number of tube junctions (*, significance using unpaired student t-test, n=3).

FIGS. 9A and 9B show LM11A-31 improves visual acuity in WT mice post I/R injury. (FIG. 9A) Follow-up chart of visual-clue water-maze test showing relative time to reach the platform (seconds) for mice undergoing I/R injury and receiving LM11A-31 for up to 15 days post I/R injury. Mice subjected to I/R injury showed significantly longer time to reach the platform starting 6 days post I/R, that was attenuated in mice receiving LM11A-31. (FIG. 9B) Overall analysis of area under the curve (AUC) after 15 days of I/R experiment. Mice subjected to I/R injury showed significantly higher AUC, that was normalized in mice receiving LM11A-31. *, two-way ANOVA showed significant interaction of disease-state (ischemia) and treatment (LM11A-31) (n=7-9).

FIG. 10 is a western blot analysis showing that diabetes significantly increases expression of p75^{NTR} in kidney from different models of diabetes including: STZ-diabetes (type-1 diabetes), db/db mice (type-2 diabetes), Akita mice (Type-1 diabetes) when compared to controls (WT) and DBA Con.

FIGS. 11A and 11B show effects of LM11A-31 on a diabetic mouse model. (FIG. 11A) Immunohistological staining of kidney section with anti-p75^{NTR} shows that diabetes (16-weeks) significantly increases expression of p75^{NTR} in kidney from diabetic animals compared to controls. (FIG. 11B) Bar graph showing an increase in Urinary albumin excretion rate (UAER), an early marker of renal injury in diabetic animals treated with LM11A-31.

FIGS. 12A, 12B, 12C are bar graphs showing the effects of LM11A-31 on (FIG. 12A) IL-6 mRNA, (FIG. 12B) IL-1β mRNA, and (FIG. 12C) urine IL-1β in a STZ-diabetes model.

FIGS. 13A-13C show interventional treatment with LM11A-31 po for 12-weeks significantly improved renal expression of fibrotic markers. (FIG. 13A) western blot showing expression levels of fibronectin and alpha SMA; (FIG. 13B) bar graph showing expression of fibronectin; (FIG. 13C) bar graph showing expression of αSMA. As used throughout, “po” means oral administration.

FIG. 14 shows interventional treatment with LM11A-31 po for 12-weeks significantly improved renal expression of TGF-β.

FIG. 15 is western blots showing interventional treatment with LM11A-31 po for 12-weeks significantly improved expression of inflammatory and apoptotic markers as well as restored NGF levels in diabetes.

FIG. 16 shows trichome histological sections from kidneys collected from various groups. Trichrome stains the muscle tissue (red), and the collagen fibers blue.

FIG. 17 shows periodic acid-Schiff (PAS) histological sections from kidneys collected from various groups. PAS stains highlight basement membranes of glomerular capillary loops and tubular epithelium.

### DETAILED DESCRIPTION

The disclosed method and compositions may be understood more readily by reference to the following detailed description of particular embodiments and the Example included therein and to the Figures and their previous and following description.

It is to be understood that the disclosed method and compositions are not limited to specific synthetic methods, specific analytical techniques, or to particular reagents unless otherwise specified, and, as such, may vary. It is also to be understood that the terminology used herein is for the purpose of describing particular embodiments only and is not intended to be limiting.

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. Thus, if a class of molecules A, B, and C are disclosed as well as a class of molecules D, E, and F and an example of a combination molecule, A-D is disclosed, then even if each is not individually recited, each is individually and collectively contemplated. Thus, is this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

### A. Definitions

It is understood that the disclosed method and compositions are not limited to the particular methodology, protocols, and reagents described as these may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a ”, “an”, and “the” include plural reference unless the context clearly dictates otherwise. Thus, for example, reference to “a MSC” includes a plurality of such MSCs, reference to “the p75^{NTR} modulator” is a reference to one or more p75^{NTR} modulators and equivalents thereof known to those skilled in the art, and so forth.

As used herein, the term “subject” or “patient” can be used interchangeably and refer to any organism to which a composition, a p75^{NTR} modulator, a MSC, or a MSC secretome of this invention may be administered, e.g., for experimental, diagnostic, and/or therapeutic purposes. Typical subjects include animals (e.g., mammals such as non-human primates, and humans; avians; domestic household or farm animals such as cats, dogs, sheep, goats, cattle, horses and pigs; laboratory animals such as mice, rats and guinea pigs; rabbits; fish; reptiles; zoo and wild animals). Typically, “subjects” are animals, including mammals such as humans and primates; and the like.

By “treat” is meant to administer a protein, nucleic acid, or composition of the invention to a subject, such as a human or other mammal (for example, an animal model), that has an increased susceptibility for developing retinal ischemia and/or diabetes or that has retinal ischemia and/or diabetes, in order to prevent or delay a worsening of the effects of the disease or condition, or to partially or fully reverse the effects of the disease or condition.

By “prevent” is meant to minimize the chance that a subject who has an increased susceptibility for developing retinal ischemia and/or diabetes actually develops the disease/condition or otherwise develops a cause of symptom thereof.

As used herein, the terms “administering” and “administration” refer to any method of providing a disclosed compositions, a p75^{NTR} modulator, a MSC, or a MSC secretome to a subject. Such methods are well known to those skilled in the art and include, but are not limited to: oral administration, transdermal administration, administration by inhalation, nasal administration, topical administration, intravaginal administration, ophthalmic administration, intraaural administration, intracerebral administration, rectal administration, sublingual administration, buccal administration, and parenteral administration, including injectable such as intravenous administration, intra-arterial administration, intramuscular administration, and subcutaneous administration. Administration can be continuous or intermittent. In various aspects, a preparation can be administered therapeutically; that is, administered to treat an existing disease or condition. In further various aspects, a preparation can be administered prophylactically; that is, administered for prevention of a disease or condition. In an aspect, the skilled person can determine an efficacious dose, an efficacious schedule, or an efficacious route of administration for a disclosed composition or a disclosed protein so as to treat a subject or induce an immune response. In an aspect, the skilled person can also alter or modify an aspect of an administering step so as to improve efficacy of a disclosed protein, nucleic acid, composition, or a pharmaceutical preparation.

As used throughout, “po” means oral administration. For example, interventional treatment with LM11A-31 po refers to oral administration of LM11A-31.

“Optional” or “optionally” means that the subsequently described event, circumstance, or material may or may not occur or be present, and that the description includes instances where the event, circumstance, or material occurs or is present and instances where it does not occur or is not present.

Ranges may be expressed herein as from “about” one particular value, and/or to “about” another particular value. When such a range is expressed, also specifically contemplated and considered disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. Finally, it should be understood that all of the individual values and sub-ranges of values contained within an explicitly disclosed range are also specifically contemplated and should be considered disclosed unless the context specifically indicates otherwise. The foregoing applies regardless of whether in particular cases some or all of these embodiments are explicitly disclosed.

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described. Publications cited herein and the material for which they are cited are hereby specifically incorporated by reference. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinence of the cited documents. It will be clearly understood that, although a number of publications are referred to herein, such reference does not constitute an admission that any of these documents forms part of the common general knowledge in the art.

Throughout the description and claims of this specification, the word “comprise” and variations of the word, such as “comprising” and “comprises,” means “including but not limited to,” and is not intended to exclude, for example, other additives, components, integers or steps. In particular, in methods stated as comprising one or more steps or operations it is specifically contemplated that each step comprises what is listed (unless that step includes a limiting term such as “consisting of”), meaning that each step is not intended to exclude, for example, other additives, components, integers or steps that are not listed in the step.

### B. p75 Modulators

In some aspects, a p75^{NTR} modulator can be a compound, nucleic acid sequence, or peptide.

In some aspects, the p75^{NTR} modulator is LM11A-31 or a derivative thereof. In some aspects, the p75^{NTR} modulator can be, but is not limited to, LM11A-24, THX-B, EVT901 or any derivatives thereof. In some aspects, the p75^{NTR} modulator is a mimetic of a neurotrophin. For example, any of the p75^{NTR} modulators described in U.S. Pat. No. 8,916,556, incorporated by reference in its entirety herein, can be used in the disclosed methods.

In some aspects, the p75^{NTR} modulator can be any compound, nucleic acid sequence, or peptide that reduces p75^{NTR} mRNA expression. For example, in some aspects, the p75^{NTR} modulator can be siRNA or antisense RNA. In some aspects, siRNA can be delivered using an expression vector. An example of a p75NTR-specific siRNA expression vector can be constructed by ligating a double-stranded hairpin oligonucleotide: 5′-GAT CCG AGG ATC GGA GGC TTG TCA TTC AAG AGA TGA CAA GCC TCC GAT CCT CTT TTT TGG AAA-3′ (SEQ ID NO:1), containing a p75^{NTR}-specific siRNA sequence (underlined), into a vector. In some aspects, the p75^{NTR} siRNA is a known siRNA from Dharmacon. In some aspects, any of the p75^{NTR} modulators disclosed herein can be used in any of the disclosed methods.

### 1. Pharmaceutical Compositions

Any of the disclosed p75^{NTR} modulators can be formulated in a composition, specifically a pharmaceutical composition. Pharmaceutical compositions can comprise a p75^{NTR} modulator and a pharmaceutically acceptable carrier.

Pharmaceutically acceptable carriers are well known to those skilled in the art and include, but are not limited to, from about 0.01 to about 0.1 M and preferably 0.05M phosphate buffer or 0.8% saline. Such pharmaceutically acceptable carriers can be aqueous or non-aqueous solutions, suspensions and emulsions.

Examples of non-aqueous solvents suitable for use in the presently disclosed subject matter include, but are not limited to, propylene glycol, polyethylene glycol, vegetable oils such as olive oil, and injectable organic esters such as ethyl oleate.

Aqueous carriers suitable for use in the presently disclosed subject matter include, but are not limited to, water, ethanol, alcoholic/aqueous solutions, glycerol, emulsions or suspensions, including saline and buffered media. Oral carriers can be elixirs, syrups, capsules, tablets and the like.

Liquid carriers suitable for use in the presently disclosed subject matter can be used in preparing solutions, suspensions, emulsions, syrups, elixirs and pressurized compounds. The active ingredient can be dissolved or suspended in a pharmaceutically acceptable liquid carrier such as water, an organic solvent, a mixture of both or pharmaceutically acceptable oils or fats. The liquid carrier can contain other suitable pharmaceutical additives such as solubilizers, emulsifiers, buffers, preservatives, sweeteners, flavoring agents, suspending agents, thickening agents, colors, viscosity regulators, stabilizers or osmo-regulators.

Liquid carriers suitable for use in the presently disclosed subject matter include, but are not limited to, water (partially containing additives as above, e.g. cellulose derivatives, preferably sodium carboxymethyl cellulose solution), alcohols (including monohydric alcohols and polyhydric alcohols, e.g. glycols) and their derivatives, and oils (e.g. fractionated coconut oil and arachis oil). For parenteral administration, the carrier can also include an oily ester such as ethyl oleate and isopropyl myristate. Sterile liquid carriers are useful in sterile liquid form comprising compounds for parenteral administration. The liquid carrier for pressurized compounds disclosed herein can be halogenated hydrocarbon or other pharmaceutically acceptable propellent.

Solid carriers suitable for use in the presently disclosed subject matter include, but are not limited to, inert substances such as lactose, starch, glucose, methyl-cellulose, magnesium stearate, dicalcium phosphate, mannitol and the like. A solid carrier can further include one or more substances acting as flavoring agents, lubricants, solubilizers, suspending agents, fillers, glidants, compression aids, binders or tablet-disintegrating agents; it can also be an encapsulating material. In powders, the carrier can be a finely divided solid which is in admixture with the finely divided active compound. In tablets, the active compound is mixed with a carrier having the necessary compression properties in suitable proportions and compacted in the shape and size desired. The powders and tablets preferably contain up to 99% of the active compound. Suitable solid carriers include, for example, calcium phosphate, magnesium stearate, talc, sugars, lactose, dextrin, starch, gelatin, cellulose, polyvinylpyrrolidine, low melting waxes and ion exchange resins.

Parenteral carriers suitable for use in the presently disclosed subject matter include, but are not limited to, sodium chloride solution. Ringer's dextrose, dextrose and sodium chloride, lactated Ringer's and fixed oils. Intravenous carriers include fluid and nutrient replenishers, electrolyte replenishers such as those based on Ringer's dextrose and the like. Preservatives and other additives can also be present, such as, for example, antimicrobials, antioxidants, chelating agents, inert gases and the like.

Carriers suitable for use in the presently disclosed subject matter can be mixed as needed with disintegrants, diluents, granulating agents, lubricants, binders and the like using conventional techniques known in the art. The carriers can also be sterilized using methods that do not deleteriously react with the compounds, as is generally known in the art.

### C. Methods of Treating Ocular Diseases

Disclosed are methods of treating an ocular disease associated with retinal ischemic injury in a subject using a p75^{NTR} modulator. In some aspects, an ocular disease associated with retinal ischemic injury can be, but are not limited to, diabetic retinopathy, retinopathy of prematurity, or traumatic neuropathy.

In some aspects, disclosed are methods of treating a retinal ischemic injury in a subject using a p75^{NTR} modulator.

In some aspects, any of the p75^{NTR} modulators disclosed herein can be used in the disclosed methods. For example, the p75^{NTR} modulator can be LM11A-31 or a derivative thereof. In some aspects, the p75^{NTR} modulator can be a p75^{NTR}-specific siRNA.

### 1. Administration of p75NTR Modulator

Disclosed are methods of treating an ocular disease associated with retinal ischemic injury in a subject comprising administering to the subject in need thereof a p75^{NTR} modulator. Disclosed are methods of treating a retinal ischemic injury in a subject comprising administering to the subject in need thereof a p75^{NTR} modulator.

In some aspects, ischemic areas on retinal angiogram are reduced. In some aspects, acellular capillaries in the subject's retina are reduced.

In some aspects, the disclosed methods of treating an ocular disease associated with retinal ischemic injury in a subject can further comprise administering to the subject a standard treatment for retinal ischemia. Therefore, disclosed are combination therapies comprising a p75^{NTR} modulator and a known treatment for retinal ischemia. In some aspects, the standard treatment for retinal ischemia is anti-vascular endothelial grown factor (anti-VEGF). In some aspects, a standard treatment for retinal ischemia can be steroids. In some aspects, the standard treatment for retinal ischemia administered in addition to the p75^{NTR} modulator can be administered via the same or a different route of administration from the p75^{NTR} modulator. For example, the p75^{NTR} modulator can be administered via oral gavage and the anti-VEGF can be administered via ocular injection.

In some aspects, the p75^{NTR} modulator is administered via oral gavage, intravitreal injection, or subconjunctival injection.

Disclosed are methods of treating an ocular disease associated with retinal ischemic injury in a subject comprising administering to the subject in need thereof a p75^{NTR} modulator, wherein the subject is not diabetic. In other words, in some aspects, the ocular disease associated with retinal ischemic injury is not diabetes.

In some aspects, a p75^{NTR} modulator results in a decrease in p75^{NTR} activity, p75^{NTR} mRNA levels, or p75^{NTR} protein levels. Therefore, in some aspects, p75^{NTR} activity, p75^{NTR} mRNA levels, or p75^{NTR} protein levels can be tested in a subject receiving a p75^{NTR} modulator. Dosing of the p75^{NTR} modulator can be increased or decreased based on the results of the subject's p75^{NTR} activity, p75^{NTR} mRNA levels, and/or p75^{NTR} protein levels.

### 2. Cell Therapy

Not only can a p75^{NTR} modulator be directly administered to a subject in need thereof either alone or formulated as a pharmaceutical composition, but a p75^{NTR} modulator can be delivered to one or more cells, in vitro or ex vivo, and then the one or more cells can be administered to a subject in need thereof. In some aspects, the cell is a mesenchymal stem cell (MSC).

Disclosed are methods of treating an ocular disease associated with retinal ischemic injury in a subject comprising administering to the subject in need thereof one or more mesenchymal stem cells (MSCs) comprising decreased p75^{NTR} expression or activity.

Disclosed are methods of treating retinal ischemic injury in a subject comprising administering to the subject in need thereof one or more MSCs comprising decreased p75^{NTR} expression or activity.

In some aspects, the decreased p75^{NTR} expression is a decrease in p75^{NTR} mRNA expression due to the p75^{NTR} modulator. For example, a p75^{NTR} modulator can be a p75^{NTR}-specific siRNA which can reduce or decrease p75^{NTR} mRNA expression.

In some aspects, the decreased p75^{NTR} expression or activity in the one or more MSCs comprising decreased p75^{NTR} expression or activity is due to a decrease in protein levels or amounts. In some aspects, the decreased p75^{NTR} activity is due to a blocking of p75^{NTR} activity in the one or more MSCs without affecting p75^{NTR} protein levels in the one or more MSCs.

In some aspects, prior to administering the subject the one or more MSCs comprising decreased p75^{NTR} expression or activity, the MSCs are treated with a p75^{NTR} modulator. In some aspects, the p75^{NTR} modulator causes a decrease in p75^{NTR} activity or expression in the MSCs. In some aspects, the decrease in p75^{NTR} activity or expression is compared to MSCs not treated with a p75^{NTR} modulator.

In some aspects, prior to administering the subject the one or more MSCs comprising decreased p75^{NTR} expression or activity, the MSCs are tested for p75^{NTR} activity. In some aspects, only those MSCs with p75^{NTR} activity of 50% of original activity are administered to the subject. In some aspects, only those MSCs with p75^{NTR} activity of 25% to 75% of original activity are administered to the subject. In some aspects, only those MSCs with p75^{NTR} expression levels of 50% of original activity are administered to the subject. In some aspects, only those MSCs with p75^{NTR} expression levels of 25% to 75% of original activity are administered to the subject.

### 3. Administration of MSC Secretome

In some aspects, neither a pharmacologic therapy or a cell therapy is used, but instead a MSC secretome is used as a therapeutic.

Disclosed are methods of treating an ocular disease associated with retinal ischemic injury in a subject comprising administering to the subject in need thereof a MSC secretome collected from one or more MSCs comprising decreased p75^{NTR} expression or activity.

Disclosed are methods of treating retinal ischemic injury in a subject comprising administering to the subject in need thereof a MSC secretome collected from one or more MSCs comprising decreased p75^{NTR} expression or activity.

In some aspects, a MSC comprising decreased p75^{NTR} expression or activity results from contacting the MSC with a p75^{NTR} modulator. Thus, in some aspects, the methods further comprise a step of contacting a MSC with a p75^{NTR} modulator prior to collecting the MSC secretome. In some aspects, the MSC secretome used in the disclosed methods is collected from MSCs previously contacted with a p75^{NTR} modulator. In some aspects, the MSC is in culture and the MSC secretome is collected from culture medium. Thus, in some aspects, contacting the MSC with a p75^{NTR} modulator can comprise adding a p75^{NTR} modulator to a cell culture comprising one or more MSCs.

In some aspects, any of the p75^{NTR} modulators disclosed herein can be used in the disclosed methods. For example, the p75^{NTR} modulator can be LM11A-31 or a derivative thereof. In some aspects, a p75^{NTR} modulator can be a p75^{NTR}-specific siRNA which can reduce or decrease p75^{NTR} mRNA expression.

In some aspects, a MSC comprising decreased p75^{NTR} expression or activity results from a genetically modified MSC to decrease expression or activity of p75^{NTR} in the MSC. In some aspects, genetically modified MSCs having decreased expression or activity of p75^{NTR} comprise a reduction or a knock down mutation of p75^{NTR} in the MSCs.

In some aspects, the decreased p75^{NTR} activity is due to a decrease in p75^{NTR} protein levels or amounts. In some aspects, the decreased p75^{NTR} activity is due to a blocking of p75^{NTR} activity without affecting p75^{NTR} protein levels.

In some aspects, prior to administering the subject the MSC secretome, the MSC secretome is tested for the presence of nerve growth factor (NGF), vascular endothelial growth factor (VEGF), and/or stromal derived factor (SDF-1). In some aspects, only those MSC secretomes with one or more of NGF, VEGF, and SDF-1 are administered to the subject.

### D. Methods of Improving Visual Acuity

Disclosed are methods of improving visual acuity in a subject having a retinal ischemic injury.

In some aspects, the disclosed methods comprise a decrease in p75^{NTR}. In some aspects, a p75^{NTR} modulator can be administered to a subject, one or more MSCs comprising decreased p75^{NTR} expression or activity can be administered to a subject, or a MSC secretome collected from one or more MSCs comprising decreased p75^{NTR} expression or activity can be administered to a subject.

In some aspects, any of the disclosed p75^{NTR} modulators can be used.

### 1. Administration of p75NTR Modulator

Disclosed are methods of improving visual acuity in a subject having a retinal ischemic injury comprising administering to the subject in need thereof a p75^{NTR} modulator.

In some aspects, a subject having a retinal ischemic injury is a subject having an ocular disease associated with retinal ischemic injury. In some aspects, an ocular disease associated with retinal ischemic injury can be, but are not limited to, diabetic retinopathy, retinopathy of prematurity, or traumatic neuropathy.

In some aspects, any of the p75^{NTR} modulators disclosed herein can be used in the disclosed methods. For example, the p75^{NTR} modulator can be LM11A-31 or a derivative thereof. In some aspects, the p75^{NTR} modulator can be a p75^{NTR}-specific siRNA.

In some aspects, improving visual acuity can be due to the p75^{NTR} modulator reducing ischemic areas on retinal angiograms. In some aspects, improving visual acuity can be due to the p75^{NTR} modulator reducing acellular capillaries in the subject's retina.

In some aspects, the p75^{NTR} modulator is administered via oral gavage, intravitreal injection, or subconjunctival injection.

In some aspects, the disclosed methods of improving visual acuity in a subject having a retinal ischemic injury comprising administering to the subject in need thereof a p75^{NTR} modulator can further comprise administering to the subject a standard treatment for retinal ischemia. Therefore, disclosed are combination therapies comprising a p75^{NTR} modulator and a known treatment for retinal ischemia. In some aspects, the standard treatment for retinal ischemia is anti-vascular endothelial grown factor (anti-VEGF). In some aspects, a standard treatment for retinal ischemia can be steroids. In some aspects, the standard treatment for retinal ischemia administered in addition to the p75^{NTR} modulator can be administered via the same or a different route of administration from the p75^{NTR} modulator. For example, the p75^{NTR} modulator can be administered via oral gavage and the anti-VEGF can be administered via ocular injection.

Disclosed are methods of improving visual acuity in a subject having a retinal ischemic injury comprising administering to the subject in need thereof a p75^{NTR} modulator, wherein the subject is not diabetic. In other words, in some aspects, the ocular disease associated with retinal ischemic injury is not diabetes.

### 2. Cell Therapy

Not only can a p75^{NTR} modulator be directly administered to a subject in need thereof, either alone or formulated as a pharmaceutical composition, but a p75^{NTR} modulator can be delivered to one or more cells, in vitro, and then the one or more cells can be administered to a subject in need thereof. In some aspects, the cell is a mesenchymal stem cell (MSC).

In some aspects, any of the p75^{NTR} modulators disclosed herein can be used in the disclosed methods. For example, the p75^{NTR} modulator can be LM11A-31 or a derivative thereof. In some aspects, the p75^{NTR} modulator can be a p75^{NTR}-specific siRNA.

Disclosed are methods of improving visual acuity in a subject having a retinal ischemic injury comprising administering to the subject in need thereof one or more MSCs comprising decreased p75^{NTR} expression or activity.

In some aspects, the decreased p75^{NTR} expression is a decrease in p75^{NTR} mRNA expression due to the p75^{NTR} modulator. For example, a p75^{NTR} modulator can be a p75^{NTR}-specific siRNA which can reduce or decrease p75^{NTR} mRNA expression.

In some aspects, the decreased p75^{NTR} expression or activity in the one or more MSCs comprising decreased p75^{NTR} expression or activity is due to a decrease in p75^{NTR} protein levels or amounts. In some aspects, the decreased p75^{NTR} activity is due to a blocking of p75^{NTR} activity in the one or more MSCs without affecting p75^{NTR} protein levels in the one or more MSCs.

In some aspects, prior to administering the subject the one or more MSCs comprising decreased p75^{NTR} expression or activity, the MSCs are treated with a p75^{NTR} modulator. In some aspects, the p75^{NTR} modulator causes a decrease in p75^{NTR} activity or expression in the MSCs. In some aspects, the decrease in p75^{NTR} activity or expression is compared to MSCs not treated with a p75^{NTR} modulator.

In some aspects, prior to administering the subject the one or more MSCs comprising decreased p75^{NTR} expression or activity, the MSCs are tested for p75^{NTR} activity. In some aspects, only those MSCs with p75^{NTR} activity of 50% of original activity are administered to the subject. In some aspects, only those MSCs with p75^{NTR} activity of 25% to 75% of original activity are administered to the subject. In some aspects, only those MSCs with p75^{NTR} expression levels of 50% of original activity are administered to the subject. In some aspects, only those MSCs with p75^{NTR} expression levels of 25% to 75% of original activity are administered to the subject.

### 3. Administration of MSC Secretome

In some aspects, neither a pharmacologic therapy or a cell therapy is used, but instead a MSC secretome is used as a therapeutic.

Disclosed are methods of improving visual acuity in a subject having a retinal ischemic injury comprising administering to the subject a MSC secretome collected from one or more MSCs comprising decreased p75^{NTR} expression or activity.

In some aspects, a MSC comprising decreased p75^{NTR} expression or activity results from contacting the MSC with a p75^{NTR} modulator. Thus, in some aspects, the methods further comprise a step of contacting a MSC with a p75^{NTR} modulator prior to collecting the MSC secretome. In some aspects, the MSC secretome used in the disclosed methods is collected from MSCs previously contacted with a p75^{NTR} modulator. In some aspects, the MSC is in culture and the MSC secretome is collected from culture medium. Thus, in some aspects, contacting the MSC with a p75^{NTR} modulator can comprise adding a p75^{NTR} modulator to a cell culture comprising one or more MSCs.

In some aspects, any of the p75^{NTR} modulators disclosed herein can be used in the disclosed methods. For example, the p75^{NTR} modulator can be LM11A-31 or a derivative thereof. In some aspects, a p75^{NTR} modulator can be a p75^{NTR}-specific siRNA which can reduce or decrease p75^{NTR} mRNA expression.

In some aspects, a MSC comprising decreased p75^{NTR} expression or activity results from a genetically modified MSC to decrease expression or activity of p75^{NTR} in the MSC. In some aspects, genetically modified MSCs having decreased expression or activity of p75^{NTR} comprise a reduction or a knock down mutation of p75^{NTR} in the MSCs.

In some aspects, the decreased p75^{NTR} activity is due to a decrease in p75^{NTR} protein levels or amounts. In some aspects, the decreased p75^{NTR} activity is due to a blocking of p75^{NTR} activity without affecting p75^{NTR} protein levels.

In some aspects, prior to administering the subject the MSC secretome, the MSC secretome is tested for the presence of NGF, VEGF, and/or SDF-1. In some aspects, only those MSC secretomes with one or more of NGF, VEGF, and/or SDF-1 are administered to the subject.

### E. Methods of Preventing Ischemia-Induced Retinal Capillary Degeneration

Disclosed are methods of preventing ischemia-induced retinal capillary degeneration in a subject.

In some aspects, the disclosed methods comprise a decrease or inhibition of p75^{NTR}. In some aspects, a p75^{NTR} modulator can be administered to a subject, one or more MSCs comprising decreased p75^{NTR} expression or activity can be administered to a subject, or a MSC secretome collected from one or more MSCs comprising decreased p75^{NTR} expression or activity can be administered to a subject.

In some aspects, any of the disclosed p75^{NTR} modulators can be used.

### 1. Administration of p75NTR Modulator

Disclosed are methods of preventing ischemia-induced retinal capillary degeneration in a subject comprising administering to the subject in need thereof a p75^{NTR} modulator, wherein the p75^{NTR} modulator inhibits the expression or activity of p75^{NTR} in mesenchymal stem cells (MSCs) in the retina of the subject.

In some aspects, any of the p75^{NTR} modulators disclosed herein can be used in the disclosed methods. For example, the p75^{NTR} modulator can be LM11A-31 or a derivative thereof. In some aspects, the p75^{NTR} modulator can be a p75^{NTR}-specific siRNA.

In some aspects, the p75^{NTR} modulator can reduce ischemic areas on retinal angiograms. In some aspects, the p75^{NTR} modulator can reduce acellular capillaries in the subject's retina.

In some aspects, the p75^{NTR} modulator is administered via oral gavage, intravitreal injection, or subconjunctival injection.

### 2. Cell Therapy

Not only can a p75^{NTR} modulator be directly administered to a subject in need thereof, either alone or formulated as a pharmaceutical composition, but a p75^{NTR} modulator can be delivered to to one or more cells, in vitro, and then the one or more cells can be administered to a subject in need thereof. In some aspects, the cell is a mesenchymal stem cell (MSC).

In some aspects, any of the p75^{NTR} modulators disclosed herein can be used in the disclosed methods. For example, the p75^{NTR} modulator can be LM11A-31 or a derivative thereof. In some aspects, the p75^{NTR} modulator can be a p75^{NTR}-specific siRNA.

Disclosed are methods of preventing ischemia-induced retinal capillary degeneration in a subject comprising administering to the subject one or more MSCs comprising decreased p75^{NTR} expression or activity.

In some aspects, the decreased p75^{NTR} expression is a decrease in p75^{NTR} mRNA expression due to the p75^{NTR} modulator. For example, a p75^{NTR} modulator can be a p75^{NTR}-specific siRNA which can reduce or decrease p75^{NTR} mRNA expression.

In some aspects, the decreased p75^{NTR} expression or activity in the one or more MSCs comprising decreased p75^{NTR} expression or activity is due to a decrease in p75^{NTR} protein levels or amounts. In some aspects, the decreased p75^{NTR} activity is due to a blocking of p75^{NTR} activity in the one or more MSCs without affecting p75^{NTR} protein levels in the one or more MSCs.

In some aspects, prior to administering the subject the one or more MSCs comprising decreased p75^{NTR} expression or activity, the MSCs are treated with a p75^{NTR} modulator. In some aspects, the p75^{NTR} modulator causes a decrease in p75^{NTR} activity or expression in the MSCs. In some aspects, the decrease in p75^{NTR} activity or expression is compared to MSCs not treated with a p75^{NTR} modulator.

In some aspects, prior to administering the subject the one or more MSCs comprising decreased p75^{NTR} expression or activity, the MSCs are tested for p75^{NTR} activity. In some aspects, only those MSCs with p75^{NTR} activity of 50% of original activity are administered to the subject. In some aspects, only those MSCs with p75^{NTR} activity of 25% to 75% of original activity are administered to the subject. In some aspects, only those MSCs with p75^{NTR} expression levels of 50% of original activity are administered to the subject. In some aspects, only those MSCs with p75^{NTR} expression levels of 25% to 75% of original activity are administered to the subject.

### 3. Administration of MSC Secretome

In some aspects, neither a pharmacologic therapy or a cell therapy is used, but instead a MSC secretome is used as a therapeutic.

Disclosed are methods preventing ischemia-induced retinal capillary degeneration in a subject comprising administering to the subject a MSC secretome collected from one or more MSCs comprising decreased p75^{NTR} expression or activity.

In some aspects, a MSC comprising decreased p75^{NTR} expression or activity results from contacting the MSC with a p75^{NTR} modulator. Thus, in some aspects, the methods further comprise a step of contacting a MSC with a p75^{NTR} modulator prior to collecting the MSC secretome. In some aspects, the MSC secretome used in the disclosed methods is collected from MSCs previously contacted with a p75^{NTR} modulator. In some aspects, the MSC is in culture and the MSC secretome is collected from culture medium. Thus, in some aspects, contacting the MSC with a p75^{NTR} modulator can comprise adding a p75^{NTR} modulator to a cell culture comprising one or more MSCs.

In some aspects, any of the p75^{NTR} modulators disclosed herein can be used in the disclosed methods. For example, the p75^{NTR} modulator can be LM11A-31 or a derivative thereof. In some aspects, a p75^{NTR} modulator can be a p75^{NTR}-specific siRNA which can reduce or decrease p75^{NTR} mRNA expression.

In some aspects, a MSC comprising decreased p75^{NTR} expression or activity results from a genetically modified MSC to decrease expression or activity of p75^{NTR} in the MSC. In some aspects, genetically modified MSCs having decreased expression or activity of p75^{NTR} comprise a reduction or a knock down mutation of p75^{NTR} in the MSCs.

In some aspects, the decreased p75^{NTR} activity is due to a decrease in p75^{NTR} protein levels or amounts. In some aspects, the decreased p75^{NTR} activity is due to a blocking of p75^{NTR} activity without affecting p75^{NTR} protein levels.

In some aspects, prior to administering the subject the MSC secretome, the MSC secretome is tested for the presence of NGF, VEGF and/or SDF-1. In some aspects, only those MSC secretomes with one or more of NGF, VEGF, and SDF-1 are administered to the subject.

### F. Methods of Increasing Vascular Protection in an Ischemic Retina

Disclosed are methods of increasing vascular protection in an ischemic retina of a subject.

In some aspects, the disclosed methods comprise a decrease in p75^{NTR}. In some aspects, a p75^{NTR} modulator can be administered to a subject, a MSC comprising decreased p75^{NTR} expression or activity can be administered to a subject, or a MSC secretome collected from a MSC comprising decreased p75^{NTR} expression or activity can be administered to a subject.

In some aspects, any of the disclosed p75^{NTR} modulators can be used.

### 1. Administration of p75NTR Modulator

Disclosed are methods of increasing vascular protection in an ischemic retina of a subject comprising administering to the retina of the subject in need thereof a p75^{NTR} modulator, wherein the p75^{NTR} modulator inhibits expression or activity of p75^{NTR} in MSCs in the retina of the subject.

In some aspects, any of the p75^{NTR} modulators disclosed herein can be used in the disclosed methods. For example, the p75^{NTR} modulator can be LM11A-31 or a derivative thereof. In some aspects, the p75^{NTR} modulator can be a p75^{NTR}-specific siRNA.

In some aspects, the p75^{NTR} modulator can reduce ischemic areas on retinal angiograms. In some aspects, the p75^{NTR} modulator can reduce acellular capillaries in the subject's retina.

In some aspects, the p75^{NTR} modulator reduces p75^{NTR} expression. In some aspects, the reduced or decreased p75^{NTR} expression is a decrease in p75^{NTR} mRNA expression due to the p75^{NTR} modulator. For example, a p75^{NTR}-specific siRNA can reduce or decrease p75^{NTR} mRNA expression.

In some aspects, the p75^{NTR} modulator is administered via oral gavage, intravitreal injection, or subconjunctival injection.

### 2. Cell Therapy

Not only can a p75^{NTR} modulator be directly administered to a subject in need thereof, either alone or formulated as a pharmaceutical composition, but a p75^{NTR} modulator can be delivered to one or more cells, in vitro, and then the one or more cells can be administered to a subject in need thereof. In some aspects, the cell is a mesenchymal stem cell (MSC).

In some aspects, any of the p75^{NTR} modulators disclosed herein can be used in the disclosed methods. For example, the p75^{NTR} modulator can be LM11A-31 or a derivative thereof. In some aspects, the p75^{NTR} modulator can be a p75^{NTR}-specific siRNA.

Disclosed are methods of increasing vascular protection in an ischemic retina of a subject comprising administering to the retina of the subject one or more MSCs comprising decreased p75^{NTR} expression or activity.

In some aspects, the decreased p75^{NTR} expression is a decrease in p75^{NTR} mRNA expression due to the p75^{NTR} modulator. For example, a p75^{NTR} modulator can be a p75^{NTR}-specific siRNA which can reduce or decrease p75^{NTR} mRNA expression.

In some aspects, the decreased p75^{NTR} activity is due to a decrease in p75^{NTR} protein levels or amounts. In some aspects, the decreased p75^{NTR} activity is due to a blocking of p75^{NTR} activity without affecting p75^{NTR} protein levels.

In some aspects, prior to administering the subject the one or more MSCs comprising decreased p75^{NTR} expression or activity, the MSCs are treated with a p75^{NTR} modulator. In some aspects, the p75^{NTR} modulator causes a decrease in p75^{NTR} activity or expression in the MSCs. In some aspects, the decrease in p75^{NTR} activity or expression is compared to MSCs not treated with a p75^{NTR} modulator.

In some aspects, prior to administering the subject the one or more MSCs comprising decreased p75^{NTR} expression or activity, the MSCs are tested for p75^{NTR} activity. In some aspects, only those MSCs with p75^{NTR} activity of 50% of original activity are administered to the subject. In some aspects, only those MSCs with p75^{NTR} activity of 25% to 75% of original activity are administered to the subject. In some aspects, only those MSCs with p75^{NTR} expression levels of 50% of original activity are administered to the subject. In some aspects, only those MSCs with p75^{NTR} expression levels of 25% to 75% of original activity are administered to the subject.

### 3. Administration of MSC Secretome

In some aspects, neither a pharmacologic therapy or a cell therapy is used, but instead a MSC secretome is used as a therapeutic.

Disclosed are methods of increasing vascular protection in an ischemic retina of a subject comprising administering to the retina of the subject a MSC secretome collected from one or more MSCs comprising decreased p75^{NTR} expression or activity.

In some aspects, a MSC comprising decreased p75^{NTR} expression or activity results from contacting the MSC with a p75^{NTR} modulator. Thus, in some aspects, the methods further comprise a step of contacting a MSC with a p75^{NTR} modulator prior to collecting the MSC secretome. In some aspects, the MSC secretome used in the disclosed methods is collected from MSCs previously contacted with a p75^{NTR} modulator. In some aspects, the MSC is in culture and the MSC secretome is collected from culture medium. Thus, in some aspects, contacting the MSC with a p75^{NTR} modulator can comprise adding a p75^{NTR} modulator to a cell culture comprising one or more MSCs.

In some aspects, any of the p75^{NTR} modulators disclosed herein can be used in the disclosed methods. For example, the p75^{NTR} modulator can be LM11A-31 or a derivative thereof. In some aspects, a p75^{NTR} modulator can be a p75^{NTR}-specific siRNA which can reduce or decrease p75^{NTR} mRNA expression.

In some aspects, a MSC comprising decreased p75^{NTR} expression or activity results from a genetically modified MSC to decrease expression or activity of p75^{NTR} in the MSC. In some aspects, genetically modified MSCs having decreased expression or activity of p75^{NTR} comprise a reduction or a knock down mutation of p75^{NTR} in the MSCs.

In some aspects, the decreased p75^{NTR} activity is due to a decrease in p75^{NTR} protein levels or amounts. In some aspects, the decreased p75^{NTR} activity is due to a blocking of p75^{NTR} activity without affecting p75^{NTR} protein levels.

In some aspects, prior to administering the subject the MSC secretome, the MSC secretome is tested for the presence of NGF, VEGF, and/or SDF-1. In some aspects, only those MSC secretomes with one or more of NGF, VEGF, and SDF-1 are administered to the subject.

### G. Methods of Increasing Vascular Homing of MSCs at a Site of Ischemic Injury

Disclosed are methods of increasing vascular homing of MSCs at a site of ischemic injury in a subject.

In some aspects, the disclosed methods comprise a decrease in p75^{NTR}. In some aspects, a p75^{NTR} modulator can be administered to a subject, one or more MSCs comprising decreased p75^{NTR} expression or activity can be administered to a subject, or a MSC secretome collected from one or more MSCs comprising decreased p75^{NTR} expression or activity can be administered to a subject.

In some aspects, any of the disclosed p75^{NTR} modulators can be used.

### 1. Administration of p75NTR Modulator

Disclosed are methods of increasing vascular homing of MSCs at a site of ischemic injury in a subject comprising administering to the subject in need thereof a p75^{NTR} modulator, wherein the p75^{NTR} modulator inhibits expression or activity of p75^{NTR} in the MSCs. In some aspects, the site of ischemic injury is in the retina of the subject.

In some aspects, the concentration of MSCs are increased in the retinal capillaries at the site of the ischemic injury in the subject's retina. In other words, the number of MSCs increase in the retinal capillaries at the site of the ischemic injury in the subject's retina in response to administering a p75^{NTR} modulator to the subject.

In some aspects, any of the p75^{NTR} modulators disclosed herein can be used in the disclosed methods. For example, the p75^{NTR} modulator can be LM11A-31 or a derivative thereof. In some aspects, the p75^{NTR} modulator can be a p75^{NTR}-specific siRNA.

In some aspects, the p75^{NTR} modulator reduces p75^{NTR} expression. In some aspects, the reduced or decreased p75^{NTR} expression is a decrease in p75^{NTR} mRNA expression due to the p75^{NTR} modulator. For example, a p75^{NTR}-specific siRNA can reduce or decrease p75^{NTR} mRNA expression.

In some aspects, the p75^{NTR} modulator is administered via oral gavage, intravitreal injection, or subconjunctival injection.

### 2. Cell Therapy

Not only can a p75^{NTR} modulator be directly administered to a subject in need thereof, either alone or

Not only can a p75^{NTR} modulator be directly administered to a subject in need thereof, either alone or formulated as a pharmaceutical composition, but a p75^{NTR} modulator can be delivered to to one or more cells, in vitro, and then the one or more cells can be administered to a subject in need thereof. In some aspects, the cell is a mesenchymal stem cell (MSC).as a pharmaceutical composition, but a p75^{NTR} modulator can be delivered to a cell, in vitro, and then the cell can be administered to a subject in need thereof.

In some aspects, any of the p75^{NTR} modulators disclosed herein can be used in the disclosed methods. For example, the p75^{NTR} modulator can be LM11A-31 or a derivative thereof. In some aspects, the p75^{NTR} modulator can be a p75^{NTR}-specific siRNA.

Disclosed are methods of increasing vascular homing of MSCs at a site of ischemic injury in a subject comprising administering to the subject one or more MSCs comprising decreased p75^{NTR} expression or activity.

In some aspects, the decreased p75^{NTR} expression is a decrease in p75^{NTR} mRNA expression due to the p75^{NTR} modulator. For example, a p75^{NTR} modulator can be a p75^{NTR}-specific siRNA which can reduce or decrease p75^{NTR} mRNA expression.

In some aspects, the decreased p75^{NTR} activity is due to a decrease in p75^{NTR} protein levels or amounts. In some aspects, the decreased p75^{NTR} activity is due to a blocking of p75^{NTR} activity without affecting p75^{NTR} protein levels.

In some aspects, prior to administering the subject the one or more MSCs comprising decreased p75^{NTR} expression or activity, the MSCs are treated with a p75^{NTR} modulator. In some aspects, the p75^{NTR} modulator causes a decrease in p75^{NTR} activity or expression in the MSCs. In some aspects, the decrease in p75^{NTR} activity or expression is compared to MSCs not treated with a p75^{NTR} modulator.

In some aspects, prior to administering the subject the one or more MSCs comprising decreased p75^{NTR} expression or activity, the MSCs are tested for p75^{NTR} activity. In some aspects, only those MSCs with p75^{NTR} activity of 50% of original activity are administered to the subject. In some aspects, only those MSCs with p75^{NTR} activity of 25% to 75% of original activity are administered to the subject. In some aspects, only those MSCs with p75^{NTR} expression levels of 50% of original activity are administered to the subject. In some aspects, only those MSCs with p75^{NTR} expression levels of 25% to 75% of original activity are administered to the subject.

### 3. Administration of MSC Secretome

In some aspects, neither a pharmacologic therapy or a cell therapy is used, but instead a MSC secretome is used as a therapeutic.

Disclosed are methods of increasing vascular homing of MSCs at a site of ischemic injury in a subject comprising administering to the subject a MSC secretome collected from one or more MSCs comprising decreased p75^{NTR} expression or activity.

In some aspects, a MSC comprising decreased p75^{NTR} expression or activity results from contacting the MSC with a p75^{NTR} modulator. Thus, in some aspects, the methods further comprise a step of contacting a MSC with a p75^{NTR} modulator prior to collecting the MSC secretome. In some aspects, the MSC secretome used in the disclosed methods is collected from MSCs previously contacted with a p75^{NTR} modulator. In some aspects, the MSC is in culture and the MSC secretome is collected from culture medium. Thus, in some aspects, contacting the MSC with a p75^{NTR} modulator can comprise adding a p75^{NTR} modulator to a cell culture comprising one or more MSCs.

In some aspects, any of the p75^{NTR} modulators disclosed herein can be used in the disclosed methods. For example, the p75^{NTR} modulator can be LM11A-31 or a derivative thereof. In some aspects, a p75^{NTR} modulator can be a p75^{NTR}-specific siRNA which can reduce or decrease p75^{NTR} mRNA expression.

In some aspects, a MSC comprising decreased p75^{NTR} expression or activity results from a genetically modified MSC to decrease expression or activity of p75^{NTR} in the MSC. In some aspects, genetically modified MSCs having decreased expression or activity of p75^{NTR} comprise a reduction or a knock down mutation of p75^{NTR} in the MSCs.

In some aspects, the decreased p75^{NTR} activity is due to a decrease in p75^{NTR} protein levels or amounts. In some aspects, the decreased p75^{NTR} activity is due to a blocking of p75^{NTR} activity without affecting p75^{NTR} protein levels.

In some aspects, prior to administering the subject the MSC secretome, the MSC secretome is tested for the presence of NGF, VEGF, and/or SDF-1. In some aspects, only those MSC secretomes with one or more of NGF, VEGF, and SDF-1 are administered to the subject.

### H. Methods of Improving MSCs Secretome

Disclosed are methods of altering a MSC's secretome comprising inhibiting the expression or activity of p75^{NTR} in the MSC. In some aspects, altering a MSC's secretome can be improving a MSC's secretome. In some aspects, an improved secretome can be a secretome comprising increased levels of NGF, VEGF, and/or SDF-1 compared to a secretome from non-treated MSC cells. In some aspects, an increase of at least 1.5 to 2-fold can be an improved secretome.

In some aspects, inhibiting the expression or activity of p75^{NTR} in the MSC comprises contacting the MSC with a p75^{NTR} modulator. In some aspects, the MSC is in culture. In some aspects, the MSC is in a subject. Thus, in some aspects, contacting the MSC with a p75^{NTR} modulator can comprise adding a p75^{NTR} modulator to a cell culture comprising one or more MSCs. In some aspects, contacting the MSC with a p75^{NTR} modulator can comprise administering a p75^{NTR} modulator to a subject, wherein the subject comprises MSCs that can come in contact with the p75^{NTR} modulator. In some aspects, the MSCs are in culture. In some aspects, the MSCs are in a retina of a subject. Therefore, the disclosed methods can be performed in vitro or in vivo.

In some aspects, any of the p75^{NTR} modulators disclosed herein can be used in the disclosed methods. For example, the p75^{NTR} modulator can be LM11A-31 or a derivative thereof. In some aspects, the p75^{NTR} modulator can be a p75^{NTR}-specific siRNA.

In some aspects, inhibiting the expression or activity of p75^{NTR} in MSCs comprises genetically modifying the MSCs to decrease expression or activity of p75^{NTR} in the MSCs. In some aspects, genetically modifying the MSCs to have decreased expression or activity of p75^{NTR} comprises a reduction or a knock down mutation of p75^{NTR} in the MSCs.

In some aspects, expression of one or more of VEGF, SDF-1α, and NGF in the MSC is increased. In some aspects, one or more of VEGF, SDF-1α, and NGF is increased in the MSC secretome. For example, if the disclosed method is performed in vitro, one or more of VEGF, SDF-1α, and NGF is increased in the cell culture medium.

### I. Methods Involving Human Retinal Endothelial Cells (HREs)

### 1. Methods of Increasing Survival Factors in Human Retinal Endothelial Cells (HREs)

Disclosed are methods of increasing survival factors in a HRE comprising contacting the HRE with a MSC having decreased p75^{NTR} expression or activity. In some aspects, levels of VEGF-A, Akt, and/or Bcl-2 in the HRE are increased.

In some aspects, contacting the HRE with the MSC having decreased p75^{NTR} expression or activity comprises first contacting the MSC with a p75^{NTR} modulator. In some aspects, any of the p75^{NTR} modulators disclosed herein can be used in the disclosed methods. For example, the p75^{NTR} modulator can be LM11A-31 or a derivative thereof. In some aspects, the p75^{NTR} modulator can be a p75^{NTR}-specific siRNA.

Disclosed are methods of increasing survival factors in a HRE comprising contacting the HRE with a MSC secretome, wherein the MSC has decreased p75^{NTR} expression or activity. In some aspects, levels of VEGF-A, Akt, and/or Bcl-2 in the HRE are increased in response to the MSC secretome. In some aspects, the MSC has decreased p75^{NTR} expression or activity due to contacting the MSC with a p75^{NTR} modulator. In some aspects, any of the p75^{NTR} modulators disclosed herein can be used in the disclosed methods. For example, the p75^{NTR} modulator can be LM11A-31 or a derivative thereof. In some aspects, the p75^{NTR} modulator can be a p75^{NTR}-specific siRNA.

In some aspects, contacting the HRE with a MSC secretome, wherein the MSC has decreased p75^{NTR} expression or activity, can comprise adding the MSC secretome to a cell culture comprising one or more HREs. In some aspects, contacting the HRE with a MSC secretome, wherein the MSC has decreased p75^{NTR} expression or activity, can comprise administering a MSC secretome, from a MSC having decreased p75^{NTR} expression or activity, to a subject, wherein the subject comprises HREs that will come in contact with the MSC secretome. In some aspects, the HREs are in culture. In some aspects, the HREs are in a retina of a subject. Therefore, the disclosed methods can be performed in vitro or in vivo.

### 2. Methods of Increasing Angiogenic Effects of HREs

Disclosed are methods of increasing angiogenic effects of a HRE comprising contacting the HRE with a MSC having decreased p75^{NTR} expression or activity, wherein HRE migration is increased.

Disclosed are methods of increasing angiogenic effects of a HRE comprising contacting the HRE with a MSC secretome, wherein the MSC has decreased p75^{NTR} expression or activity, wherein HRE migration is increased.

In some aspects, the HRE is in culture. In some aspects, the HRE is in a subject. In some aspects, contacting the HRE with one or more MSCs having decreased p75^{NTR} expression or activity comprises first contacting the MSC with a p75^{NTR} modulator. In some aspects, the decreased expression or activity of p75^{NTR} in MSCs comprises genetically modifying the MSCs to decrease expression or activity of p75^{NTR} in the MSCs. In some aspects, genetically modifying the MSCs to have decreased expression or activity of p75^{NTR} comprises a reduction or a knock down mutation of p75^{NTR} in the MSCs.

In some aspects, contacting the HRE with a MSC secretome, wherein the MSC has decreased p75^{NTR} expression or activity, can comprise administering a MSC having decreased p75^{NTR} expression or activity to a subject, wherein the subject comprises HREs that can come in contact with the MSC. In some aspects, the HREs are in culture. In some aspects, the MSCs are in a retina of a subject. Therefore, the disclosed methods can be performed in vitro or in vivo.

In some aspects of the disclosed methods, the ability of the HRE to form tubes is increased. In other words, capillaries can be formed which increases the angiogenic effect. In some aspects, one or more HREs comprise a tube, and wherein the tube length is increased in the HRE. The increase in tube length can be compared to an HRE not contacted with a MSC having decreased p75^{NTR} expression or activity or with a MSC secretome from a MSC having decreased p75^{NTR} expression or activity.

In some aspects, any of the p75^{NTR} modulators disclosed herein can be used in the disclosed methods. For example, the p75^{NTR} modulator can be LM11A-31 or a derivative thereof. In some aspects, the p75^{NTR} modulator can be a p75^{NTR}-specific siRNA.

### J. Methods of Administering a p75^{NTR} Modulator to a Subject Having Diabetes

### 1. Administration of p75NTR Modulator

Disclosed are methods of administering a p75^{NTR} modulator to a subject having diabetes. In some aspects, the subject having diabetes has advanced stage diabetes. In some aspects, advanced stage diabetes can also be known as advanced stage of diabetic complications. Advanced stage of diabetic complications can be identified by an altered (increased) ratio of albumin excretion to creatinine and low glomerular filtration rate (GFR). Histopathological changes (e.g. glomerular size and PASH stain) can also be identified. For example, late stage, or advanced stage, diabetes can be identified by diabetic kidney disease progression to renal failure. In some aspects, diabetes can cause microvascular complications such as diabetic retinopathy (eye), nephropathy (kidney) that can progress to end stage renal disease (ESRD) and renal failure.

In some aspects, any of the p75^{NTR} modulators disclosed herein can be used in the disclosed methods. For example, the p75^{NTR} modulator can be LM11A-31 or a derivative thereof. In some aspects, the p75^{NTR} modulator can be a p75^{NTR}-specific siRNA.

Disclosed are methods of decreasing urinary albumin excretion rate in a subject having diabetes comprising administering to the subject a p75^{NTR} modulator.

Disclosed are methods of decreasing inflammation in the kidney of a subject having diabetes comprising administering to the subject a p75^{NTR} modulator. In some aspects, the presence of IL-1β is decreased in the kidney or urine of the subject and/or the presence of IL-6 is decreased in the kidney of the subject and/or the presence of TNF-α is decreased in the kidney of the subject and/or the presence of NGF is increased in the kidney of the subject. The presence or absence of one or more of these, as described herein, is indicative of a decrease in inflammation in the kidney. In some aspects, IL-1β, TNF-α, and/or IL-6 are detected in the urine or blood of a subject. Because the kidney cannot necessarily be accessed in a live subject, the presence of IL-1β, TNF-α, and/or IL-6 in the urine or blood of the subject can be indicative of increased levels in the kidney.

Disclosed are methods of decreasing IL-1β in a subject having diabetes comprising administering to the subject a p75^{NTR} modulator, wherein IL-1β is decreased in one or both kidneys after administration of the p75^{NTR} modulator. In some aspects, a decrease in IL-1β is indicative of a decrease in inflammation in the kidney of the subject. In some aspects, the decrease in IL-10 is a decrease in mRNA levels, protein levels, or protein activity. In some aspects, the levels or activity of IL-1β is detected in the urine of a subject and correlates to kidney levels or activity.

Disclosed are methods of decreasing IL-6 in a subject having diabetes comprising administering to the subject in need thereof a p75^{NTR} modulator, wherein IL-6 is decreased in one or both kidneys after administration of the p75^{NTR} modulator. In some aspects, a decrease in IL-6 is indicative of a decrease in inflammation in the kidney of the subject. In some aspects, the decrease in IL-6 is a decrease in mRNA levels, protein levels, or protein activity. In some aspects, the levels or activity of IL-6 is detected in the urine of a subject and correlates to kidney levels or activity.

Disclosed are methods of decreasing TGF-β in a subject having diabetes comprising administering to the subject in need thereof a p75^{NTR} modulator, wherein TGF-β is decreased in one or both kidneys after administration of the p75^{NTR} modulator. In some aspects, a decrease in TGF-β is indicative of a decrease in inflammation in the kidney of the subject. In some aspects, the decrease in TGF-β is a decrease in mRNA levels, protein levels, or protein activity. In some aspects, the levels or activity of TGF-β is detected in the urine of a subject and correlates to kidney levels or activity.

Disclosed are methods of decreasing one or more markers of fibrosis in a subject having diabetes comprising administering to the subject in need thereof a p75NTR modulator, wherein one or more markers of fibrosis is decreased in one or both kidneys after administration of the p75NTR modulator. In some aspects, the disclosed methods of decreasing one or more markers of fibrosis can be performed in non-diabetic subjects. Thus, also disclosed are methods of decreasing one or more markers of fibrosis in a non-diabetic subject comprising administering to the subject in need thereof a p75NTR modulator, wherein one or more markers of fibrosis is decreased in one or both kidneys after administration of the p75NTR modulator. In some aspects, the markers of fibrosis are one or more of fibronectin or α-SMA. In some aspects, the markers of fibrosis are detected in the urine of a subject and correlates to kidney levels or activity.

Disclosed are methods of decreasing fibrotic tissue in a kidney of a subject having diabetes comprising administering to the subject in need thereof a p75^{NTR} modulator.

Disclosed are methods of treating late stage diabetes in a subject comprising administering to the subject in need thereof a p75^{NTR} modulator.

### 2. Cell Therapy

Not only can a p75^{NTR} modulator be directly administered to a subject in need thereof, either alone or formulated as a pharmaceutical composition, but a p75^{NTR} modulator can be delivered to one or more cells, in vitro, and then the one or more cells can be administered to a subject in need thereof. In some aspects, the cell is a mesenchymal stem cell (MSC).

In some aspects, any of the p75^{NTR} modulators disclosed herein can be used in the disclosed methods. For example, the p75^{NTR} modulator can be LM11A-31 or a derivative thereof. In some aspects, the p75^{NTR} modulator can be a p75^{NTR}-specific siRNA.

Instead of administering a p75^{NTR} modulator to a subject having diabetes in any of the disclosed methods, the methods can comprise administering to the subject having diabetes one or more MSCs comprising decreased p75^{NTR} expression or activity.

In some aspects, the decreased p75^{NTR} expression is a decrease in p75^{NTR} mRNA expression due to the p75^{NTR} modulator. For example, a p75^{NTR} modulator can be a p75^{NTR}-specific siRNA which can reduce or decrease p75^{NTR} mRNA expression.

In some aspects, the decreased p75^{NTR} activity is due to a decrease in p75^{NTR} protein levels or amounts. In some aspects, the decreased p75^{NTR} activity is due to a blocking of p75^{NTR} activity without affecting p75^{NTR} protein levels.

In some aspects, prior to administering the subject the one or more MSCs comprising decreased p75^{NTR} expression or activity, the MSCs are treated with a p75^{NTR} modulator. In some aspects, the p75^{NTR} modulator causes a decrease in p75^{NTR} activity or expression in the MSCs. In some aspects, the decrease in p75^{NTR} activity or expression is compared to MSCs not treated with a p75^{NTR} modulator.

In some aspects, prior to administering the subject the one or more MSCs comprising decreased p75^{NTR} expression or activity, the MSCs are tested for p75^{NTR} activity. In some aspects, only those MSCs with p75^{NTR} activity of 50% of original activity are administered to the subject. In some aspects, only those MSCs with p75^{NTR} activity of 25% to 75% of original activity are administered to the subject. In some aspects, only those MSCs with p75^{NTR} expression levels of 50% of original activity are administered to the subject. In some aspects, only those MSCs with p75^{NTR} expression levels of 25% to 75% of original activity are administered to the subject.

### 3. Administration of MSC Secretome

In some aspects, neither a pharmacologic therapy or a cell therapy is used, but instead a MSC secretome is used as a therapeutic.

Instead of administering a p75^{NTR} modulator or a cell therapy to a subject having diabetes in any of the disclosed methods, the methods can comprise administering to the subject a MSC secretome collected from one or more MSCs comprising decreased p75^{NTR} expression or activity.

In some aspects, a MSC comprising decreased p75^{NTR} expression or activity results from contacting the MSC with a p75^{NTR} modulator. Thus, in some aspects, the methods further comprise a step of contacting a MSC with a p75^{NTR} modulator prior to collecting the MSC secretome. In some aspects, the MSC secretome used in the disclosed methods is collected from MSCs previously contacted with a p75^{NTR} modulator. In some aspects, the MSC is in culture and the MSC secretome is collected from culture medium. Thus, in some aspects, contacting the MSC with a p75^{NTR} modulator can comprise adding a p75^{NTR} modulator to a cell culture comprising one or more MSCs.

In some aspects, any of the p75^{NTR} modulators disclosed herein can be used in the disclosed methods. For example, the p75^{NTR} modulator can be LM11A-31 or a derivative thereof. In some aspects, a p75^{NTR} modulator can be a p75^{NTR}-specific siRNA which can reduce or decrease p75^{NTR} mRNA expression.

In some aspects, a MSC comprising decreased p75^{NTR} expression or activity results from a genetically modified MSC to decrease expression or activity of p75^{NTR} in the MSC. In some aspects, genetically modified MSCs having decreased expression or activity of p75^{NTR} comprise a reduction or a knock down mutation of p75^{NTR} in the MSCs.

In some aspects, the decreased p75^{NTR} activity is due to a decrease in p75^{NTR} protein levels or amounts. In some aspects, the decreased p75^{NTR} activity is due to a blocking of p75^{NTR} activity without affecting p75^{NTR} protein levels.

In some aspects, prior to administering the subject the MSC secretome, the MSC secretome is tested for the presence of NGF, VEGF, and/or SDF-1. In some aspects, only those MSC secretomes with one or more of NGF, VEGF, and SDF-1 are administered to the subject.

### K. Administration

The presently disclosed subject matter discloses methods of administering p75^{NTR} modulators to a subject or administering p75^{NTR} modulators to a cell, wherein the cell is administered to a subject. The method can comprise the step of administering to a subject an effective amount of a composition comprising a p75^{NTR} modulator, such as any of the compositions disclosed herein.

As used herein, administering can be effected or performed using any of the various methods known to those skilled in the art. The composition can be administered, for example, subcutaneously, intravenously, parenterally, intraperitoneally, intradermally, intramuscularly, topically, enteral (e.g., orally), rectally, nasally, buccally, sublingually, vaginally, by inhalation spray, by drug pump or via an implanted reservoir in dosage formulations containing conventional non-toxic, physiologically acceptable carriers or vehicles. In some aspects, the composition can be administered via oral gavage.

Further, the presently disclosed compositions can be administered to a localized area in need of treatment. This can be achieved by, for example, and not by way of limitation, local infusion during surgery, topical application, transdermal patches, by injection, by catheter, by suppository, or by implant (the implant can optionally be of a porous, non-porous, or gelatinous material), including membranes, such as sialastic membranes or fibers.

The form in which the composition is administered (e.g., syrup, elixir, capsule, tablet, solution, foams, emulsion, gel, sol) will depend in part on the route by which it is administered. For example, for mucosal (e.g., oral mucosa, rectal, intestinal mucosa, bronchial mucosa) administration, nose drops, aerosols, inhalants, nebulizers, eye drops or suppositories can be used. The composition can also be used to coat bioimplantable materials to enhance neurite outgrowth, neural survival, or cellular interaction with the implant surface. The compositions and agents disclosed herein can be administered together with other biologically active agents, such as analgesics, anti-inflammatory agents, anesthetics and other agents which can control one or more symptoms or causes of a p75^{NTR}-mediated condition.

Additionally, administration can comprise administering to the subject a plurality of dosages over a suitable period of time. Such administration regimens can be determined according to routine methods, upon a review of the instant disclosure.

### EXAMPLES

### A. Example 1: Modulation of p75^{NTR} on Mesenchymal Stem Cells Increases Their Vascular Protection in Retinal Ischemia-Reperfusion Mouse Model

### 1. Introduction

Cell therapy in general and mesenchymal stem cells (MSCs) can be an attractive candidate for retinal regeneration. MSCs are multipotent stem cells present in adult marrow and have the potential to differentiate into lineages of mesenchymal tissues, including bone, cartilage, fat, tendon, muscle and marrow stroma. One advantage of adult MSCs is that they can be isolated from the bone marrow, peripheral blood, or adipose tissue of a subject in reasonable quantity; however, the in vitro expansion of MSCs, a lengthy process, prior to administration, can result in a loss of stemness and can be hampered by the age and the disease state of the patient. MSCs are perceived as “immune-privileged” cells and as such are widely used clinically in allogeneic settings. MSCs are home to sites of inflammation where they secrete a variety of soluble factors, including growth factors, cytokines, and chemokines. While the secretion of paracrine factors by MSCs is well-established as a reparative mechanism, the engrafting of MSCs to injured tissues has not been proven to be a perquisite for the reparative action. Animal studies demonstrated that the subretinal transplantation of MSCs delays retinal neurodegeneration and preserves neural retinal function; however, the vascular protection of MSCs transplanted in the ischemic retina has not been fully elucidated.

### 2. Results

### i. Deletion of p75^{NTR} Prevented Retinal Capillary Degeneration in Ischemia/Reperfusion Model

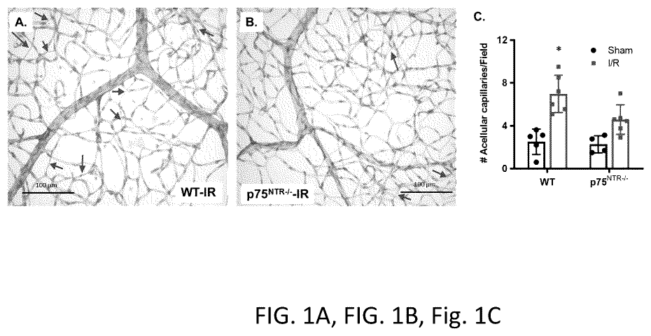

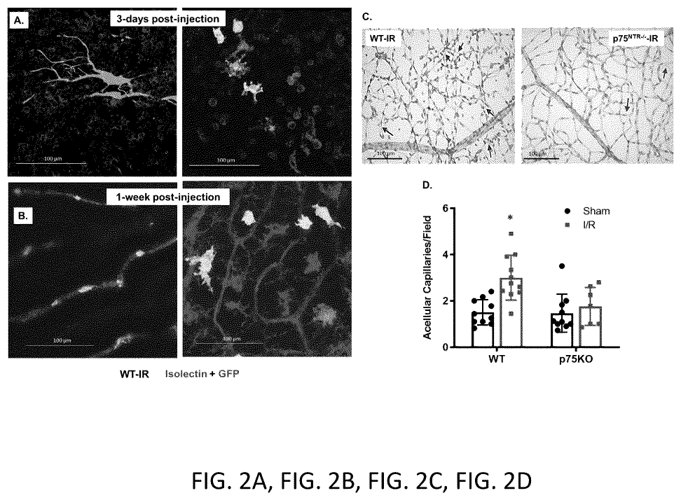

Genetic deletion of p75^{NTR} prevents diabetes-induced degeneration of retinal capillaries identified by the presence of acellular capillaries. Based on this similarity between ischemia/reperfusion (I/R) and diabetic retinopathy, the vascular protective effects of p75^{NTR} genetic deletion was examined in acute retinal ischemic/reperfusion (I/R) injury. Trypsin-digested retinas showed that I/R resulted in a significant increase in the number of acellular capillaries (3-Fold) in WT mice, compared to their sham-operated controls, whereas the sham group showed a mean of 2.51±0.5 acellular capillaries, as compared to the I/R group, showing a mean of 7.06±0.83 acellular capillaries/field (FIG. 1A, FIG. 1C). The deletion of the p75^{NTR} receptor significantly reduced the number of acellular capillaries in the ischemic retina by 35%, compared to the ischemic WT retinas. Two-way ANOVA showed the significant effects of disease state (I/R) and gene deletion. There was no significant difference between p75^{NTR-/-} in ischemic retinas when compared to the p75^{NTR-/-} sham group (FIG. 2B, FIG. 2C).

### ii. Mesenchymal Stem Cells (MSCs) Are Engrafted to Retina Capillaries and Improve Vascular Protection in Both WT and p75NTR−/− Mice

MSCs are widely used; however, whether the incorporation and engrafting of MSCs to injured tissues is required to exert their vascular protection remains to be elucidated. GFP-labelled MSCs that could be traced post intravitreal injection were used. I/R was performed and, two-days later, GFP-MSCs were intravitreally injected into WT ischemic retinas. The homing and integration of MSCs into ischemic vasculature were followed after an additional 3 days (FIG. 2A) and 1 week (FIG. 2B) post injection. Isolectin GS-stained WT retinal flat mounts showed the presence of GFP-labeled MSCs (green) as satellite-shaped within the capillary network (red), with no integration (FIG. 2A). On the other hand, at one week post intravitreal injection, GFP-MSCs showed engrafting and incorporation (yellow) within retinal capillaries (FIG. 2B). The injection of MSCs enhanced vascular protection against the formation of acellular capillaries following retinal ischemia both in WT and p75NTR−/− retinas (FIG. 2C, FIG. 2D). WT ischemic retinas that received MSCs still showed a significant 2-Fold increase in the count of acellular capillaries (3.0±0.29), as compared to the WT sham group (1.51±0.17). In contrast, the ischemic p75NTR−/− retinas that received MSCs showed a similar count for acellular capillaries (1.76±0.31) to the p75NTR−/− sham group (1.56±0.26) and was significantly lower than the count for WT ischemic retinas (3.0±0.29, FIG. 2C, FIG. 2D).

### iii. Silencing p75^{NTR} Expression in MSCs Increased Their Homing and Vascular Protection in Ischemic Retinas

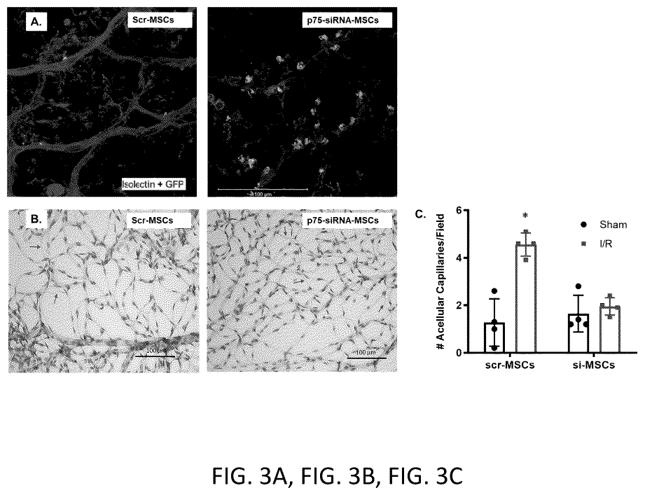

Since the p75^{NTR} has been shown to possibly inhibit differentiation of MSCS into multiple cell types, the impact of silencing p75^{NTR} on MSCs homing and engrafting into ischemic retinas was examined. Optimization experiments showed that 100 nM of siRNA against p75^{NTR} was as effective as 300 nM of siRNA to reduce p75^{NTR} mRNA expression. To assess the change in vascular homing upon knocking-down p75^{NTR} expression, the Scr- or Si-100-treated GFP-labeled MSCs were injected intravitreally, 2 days post I/R injury in WT mice (FIG. 3). Isolectin GS-stained retinal flat mounts showed enhanced recruitment and homing of GFP-labeled MSCs (green) into WT-retinal capillaries (red) after silencing p75^{NTR} expression compared to scrambled cells (FIG. 3A). Trypsin-digestion of ischemic WT retinas that received scrambled-p75^{NTR}-MSCs still showed a significant 3.3-Fold increase in the number of acellular capillaries (4.55±0.24) when compared to their sham group (1.27±0.49, FIG. 3B, FIG. 3C). Meanwhile, knocking down p75^{NTR} expression on MSCs resulted in almost complete vascular protection of ischemic WT retinas, where there was no significant difference between ischemic group and their shams receiving siRNA-MSC (1.95±0.18 versus 1.65±0.38). The number of acellular capillaries in ischemic retinas receiving siRNA-p75^{NTR}-MSCs significantly decreased by ˜58%, when compared to ischemic retinas receiving scrambled-p75^{NTR}-MSCs; FIG. 3B, FIG. 3C).

### iv. Silencing p75^{NTR} Expression on MSCs Improves Their Secretome

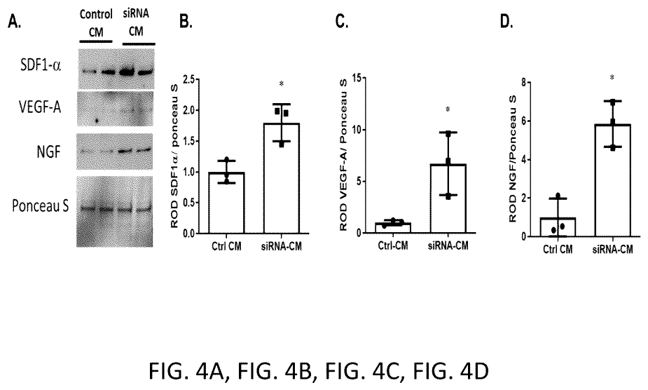

Secretion of paracrine factors by MSCs is well-established as their primary reparative mechanism. Thus, the impact of modulating p75^{NTR} on the MSCs secretome was characterized. As shown in FIG. 4, silencing p75^{NTR} expression on MSCs significantly increased the protein expression of VEGF (6 Fold), SDF-1α (1.8 Fold) and NGF (5.8 Fold) in MSCs conditioned medium (CM) when compared to control CM.

### v. Silencing p75^{NTR} Expression on MSCs Improves Paracrine Effect on HREs

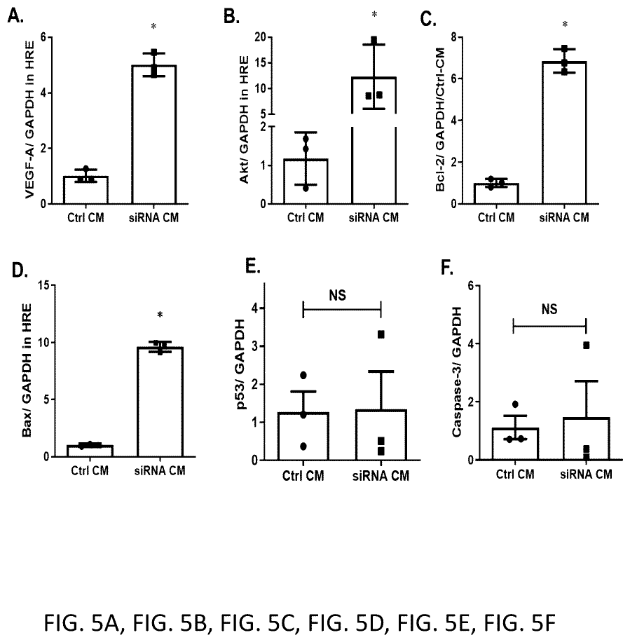

Since an improved MSC secretome was observed by silencing p75^{NTR} expression on MSCs (FIG. 4), the paracrine effect of MSCs CM on HREs was examined. As shown in FIG. 5, the treatment of HREs with CM of p75^{NTR}-silenced MSCs increased gene expression of the anti-apoptotic markers VEGF-A by 5 Fold (5.01±0.24 versus 1.02±0.13 for controls, FIG. 5A), Akt by ˜10 Fold (12.30±3.6 versus 1.18±0.39 for controls; FIG. 5B), Bcl-2 by ˜6.8 Fold (6.86±0.33 versus 1.01±0.11 for controls; FIG. 5C). For the apoptotic markers, silencing p75^{NTR} resulted in increases in Bax expression by ˜9.6 Fold (9.65±0.21 versus 1.01±0.09 for controls; FIG. 5D), however there was no change in p53 (1.27±0.54 vs. 1.35±0.98; FIG. 5E) or caspase-3 expression (1.1±0.4 vs. 1.4±1.8; FIG. 5F).

### vi. Silencing p75^{NTR} Expression on MSCs Enhanced Angiogenic Response in HREs

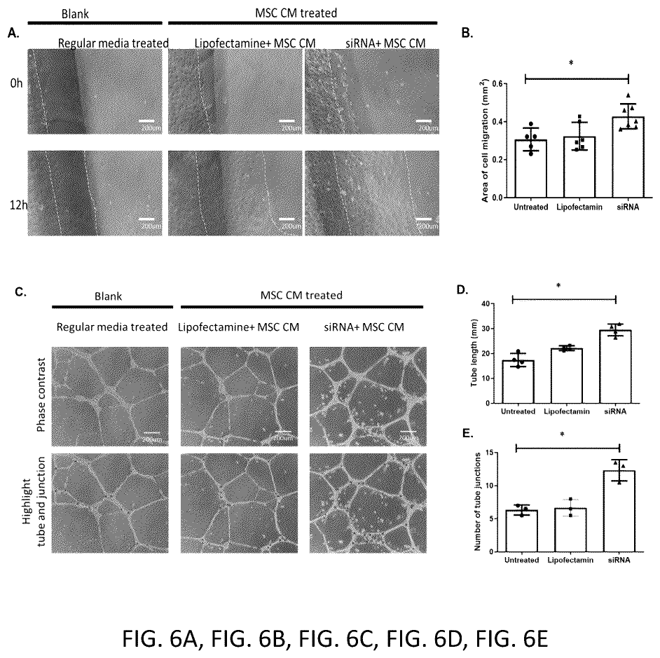

The angiogenic behavior of HREs showed significant improvement upon treatment with CM of p75^{NTR}-silenced MSCs, where HREs migration significantly increased by 1.4 Fold after 12 h of treatment (FIG. 6A, FIG. 6B). In addition, the ability of HREs to form tubes in vitro was significantly enhanced following 24 h of treatment, in terms of tube length that showed an increase of 1.5 Fold and the number of junction points that showed an increase of 2 Fold (FIGS. 6C-E).

### vii. Modulating p75^{NTR} on MSCs Using LM11A-31 Improved Their Secretome and Improved Paracrine Effect in HREs

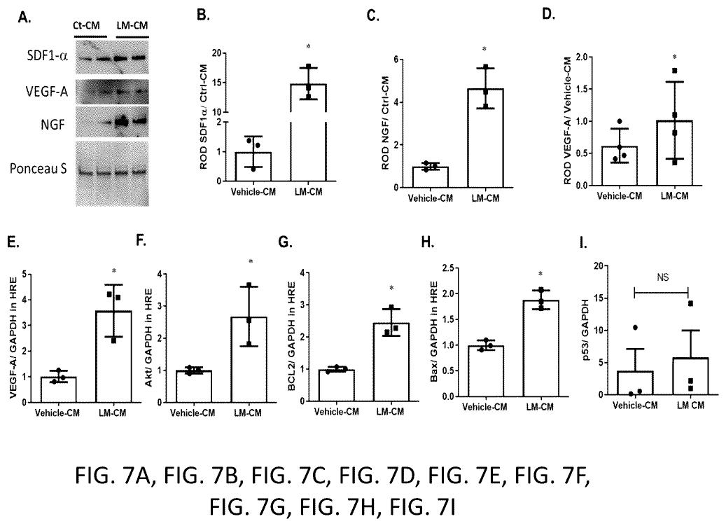

LM11A-31, the pharmacologic modulator of p75^{NTR} receptor, showed similar results to p75^{NTR} genetic silencing in improving the secretome of MSCs. As shown in FIGS. 7A-D, the treatment of MSCs with LM11A-31 increased the release of SDF-1a, VEGF-A and NGF in their CM. SDF-1a increased by ˜14.9 Fold, VEGF-A increased by 1.6 Fold and NGF increased by ˜4 Fold when compared to control CM. As shown in FIGS. 7E-I, the treatment of HREs with conditioned medium of LM11A-31-treated MSCs increased the gene expression of VEGF-A by ˜3.5 Fold (3.58±0.58 versus 1.017±0.129 for controls; FIG. 7E), Akt by ˜2.7 Fold (2.68±0.53 versus 1.0±0.06 for controls; FIG. 7F), Bcl-2 by ˜2.5 Fold (2.45±0.24 versus 1.0±0.04 for controls; FIG. 7G). For some of the apoptotic markers, CM of LM11A-31-treated MSCs increased gene expression of Bax by ˜1.9 Fold (1.88±0.105 versus 1.0±0.055 for controls; FIG. 7H), but there was no change in the expression of p53 (3.7±3.4 vs. 5.7±4.2; FIG. 7I).

### viii. Conditioned Medium of LM11A-31-Treated MSCs Enhanced Angiogenic Response in HREs

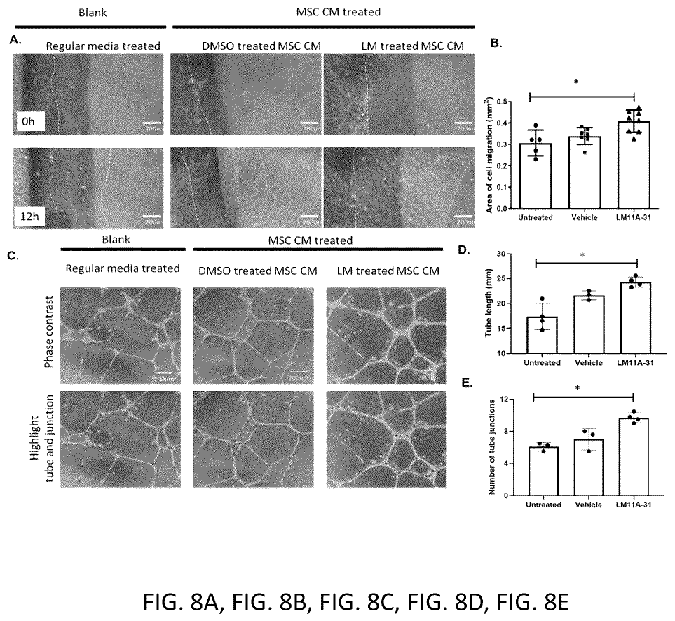

CM of p75^{NTR}-modulated MSCs using LM11A-31 showed similar enhanced angiogenic response in HREs (FIG. 8) using p75^{NTR}-siRNA (FIG. 6). HREs migration significantly increased by 1.34 Fold after 12 h of treatment (FIG. 8A, FIG. 8B). Further, the ability of HREs to form tubes in vitro was significantly enhanced following 24 h of treatment, evident by 1.4 Fold increase in tube length and 1.5 Fold increase in number of junction points when compared to controls (FIGS. 8C-E).

### ix. Modulating p75^{NTR} Using LM11A-31 Improves Visual Acuity in WT Mice Post I/R Injury

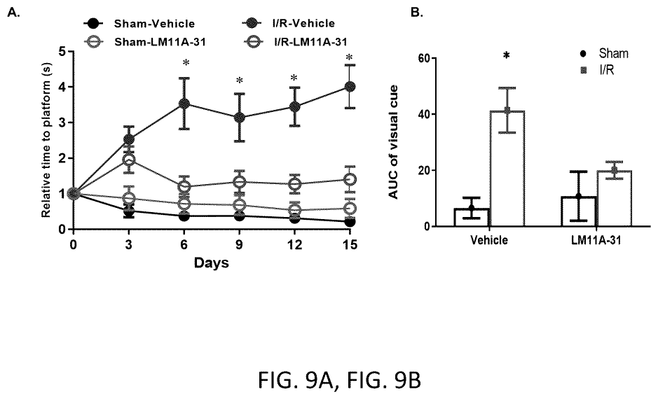

As shown in FIG. 9, mice subjected to I/R injury showed deteriorated visual function in the visual-clue water maze test, where time to reach the platform was significantly increased in mice that underwent I/R injury after 6 days, 9 days, 12 days and 15 days post I/R, an effect that was significantly ameliorated in mice receiving LM11A-31 at all tested time points.

### 3. Discussion

Retinal ischemia is a common underlying pathology for multiple retinal diseases, including diabetic retinopathy, retinopathy of prematurity, traumatic optic neuropathy and acute closed angle glaucoma. Vascular cell death and development of acellular capillaries are well-accepted surrogate markers for retinal ischemia. Stem cell therapy, in general, and mesenchymal stem cells (MSCs), in particular, have demonstrated hopeful results to improve ischemic and neuronal disorders. It has been demonstrated that increases in the neurotrophin receptor p75NTR are closely linked to retinal inflammation, barrier dysfunction and the development of acellular capillaries. Here, it is demonstrated that the vascular protective effects of modulating p75NTR and MSCs injection using an acute model of retinal ischemia/reperfusion in mice. The main findings of the current study are: (1) the vascular protective effects of p75NTR deletion against retinal ischemia is enhanced by MSCs injection; (2) silencing p75NTR expression on MSCs enhanced their vascular homing and potentiated the vascular protection against I/R-injury; (3) modulating p75NTR on MSCs using siRNA or pharmacologically using LM11A-31 enriched their secretome with trophic and angiogenic factors, such as NGF, SDF-1, and VEGF; (4) conditioned medium of p75NTR modulated MSCs stimulated the survival and angiogenic behavior of retinal endothelial cells; (5) further intervention with LM11A-31 significantly improved decline in visual acuity post retinal ischemic injury.

P75NTR signal transduction pathways are extremely variable because they are dependent on cell type, cell differentiation status, neurotrophin binding, interacting transmembrane co-receptor as well as the availability of intracellular adaptor molecule. This leads to divergent cellular responses, including progenitor differentiation, cell survival, apoptosis, and cell migration and invasion. These findings showed that transient exposure to I/R resulted in a significant increase in acellular capillaries formation in WT mice that was ameliorated in p75NTR−/− mice (FIG. 1). In support, prior study showed that vascular cell death and the development of acellular capillaries are associated with increases in the expression of p75NTR and its co-receptor sortilin post retinal I/R-injury. The vascular protection associated with p75NTR modulation have been attributed to the inhibition of inflammation and cell death signaling in capillary cells.

Capillary degeneration associated with p75NTR signaling may be due to failure of endothelial hematopoietic stem cells to maintain normal retinal vasculature or to repair damaged vasculature. The P75NTR receptor, also known as CD271, enriches several progenitor/stem cells subtypes, including MSCs. P75NTR is involved in MSCs differentiation where expression of p75NTR was shown to rapidly downregulate upon differentiation of MSCs in vitro. Moreover, p75NTR could directly hinder the differentiation of MSCs through the inhibition of transcription factors, including Runx2 and OSX, which are essential for osteoblast differentiation and for the expression of chondrogenesis marker; Sox9 and the myogenic marker, Myf5. The transplantation of bone marrow-derived MSCs was shown to rescue photoreceptor cells in dystrophic retina of rhodopsin knockout mice, indicating a therapeutic benefit in retinitis pigmentosa. The neuroprotection of MSCs has been reported in rat retina subjected to I/R, where injection of MSCs preserved number of RGCs compared to controls. While MSCs-associated neuroprotection has been demonstrated, this study explored the MSCs-mediated vascular protection in ischemic retina. The intravitreal injection of MSCs 2 days post I/R injury exerted vascular protection in both WT and p75NTR−/− mice with enhanced homing and integration into retinal capillaries (FIG. 2). Interestingly, silencing p75NTR expression on the surface of MSCs resulted in increased vascular homing of MSCs and potentiated the vascular protection evident by significant decreases in the number of acellular capillaries (FIG. 3).

To explore the possible behavior of MSCs post injection and its interaction with retinal microvasculature, a large number of MSCs (100,000 cells/eye) were injected. It was interesting to observe different behavior at different time points (FIG. 2A, FIG. 2B). At the 3 day time point, some of the MSCs morphed into a satellite shape and remained green, as they were not with the same plane of retinal capillaries (FIG. 2A: left). Other MSCs morphed into yellow, ameboid-shaped cells that co-stained with isolectin (FIG. 2A: right). Interestingly, 1 week post injection, some MSCs integrated into perivascular and wrapped around retinal capillaries (red) but remained green (FIG. 2B: Left), while others appeared yellow and retained the ameboid shape and isolectin co-staining (FIG. 2B: Right). These observations confirm the recruitment and homing of MSCs to the ischemic retina vasculature. The co-staining of GFP-MSCs with isolectin (yellow) indicates possible interaction with microglia and modulation of proinflammatory phenotype, as recently shown. The perivascular appearance raises the possibility of MSCs being differentiated into perivascular cells, such as pericytes. Pericytes have mesenchymal origin and are traditionally recruited to support endothelial tubes. The findings lend further support to the potential differentiation of injected MSCs into pericytes, as illustrated previously.

To investigate the underlying mechanism behind the augmented vascular protection observed after silencing p75NTR expression on MSCs, their CM were examined for secreted trophic and growth factors. The silencing of MSCs-p75NTR enriched their CM with VEGF, NGF and SDF-1α (FIG. 4), which coincided with enhanced HREs survival (FIG. 5) and paracrine angiogenic response (FIG. 6). There is also a tendency for increased gene expression of SDF-1α receptors, CXCR-4 and CXCR-7. SDF-1 is a classical chemoattractant stimulus of endothelial progenitor cells to endothelial lineage that is known to upregulate following ischemic insult. In agreement with these findings, MSCs overexpressing SDF-1 were shown to have increased expression of VEGF, Akt and eNOS. In vivo, they enhanced angiogenesis and improved heart function in an experimental myocardial infarction rat model. In addition, SDF-1 was shown to mediate the regenerative effects of MSCs, where knocking down MSCs-SDF-1 significantly reduced their beneficial effects on alveolarization, angiogenesis, and inflammation in a rodent model of bronchopulmonary dysplasia. It is well known that the transendothelial migration of MSCs is enhanced through the SDF-1/CXCR4 axis. The SDF-1/CXCR4 axis has been shown to improve the recruitment and hematopoietic reconstitution of bone marrow-derived MSCs in aplastic anemia. Recently, atypical chemokine receptor ACKR3, previously known as CXCR7, was also shown to promote the proliferation and migration of MSCs, as well as their secreted SDF-1, improving their treatment effectiveness. Moreover, SDF-1/CXCR-7 was reported to enhance the migration of MSCs in a transient cerebral I/R rat hippocampus model. Thus, enhanced in vivo vascular integration and protection, as well as in vitro paracrine angiogenic potential of HREs induced by silencing MSCs-p75NTR can be attributed, at least in part, to SDF-1/CXCR-4 and-7 signaling axis. VEGF and Akt are known regulators of endothelial cell survival and angiogenesis. In vitro, the treatment of HREs with enriched CM of p75NTR-silenced MSCs improved HREs survival, evident by the increased gene expression of VEGF and Akt (FIG. 5) as well as their angiogenic potential in vitro (FIG. 6). MSCs-secreted VEGF mediates the differentiation of endothelial progenitor cells into endothelial cells via paracrine mechanisms. MSCs-secreted VEGF was also shown to play a part in underlying paracrine and neuroprotective action of MSCs. NGF is another potential trophic factor that can contribute to enhanced reparative potential of p75NTR-silenced MSCs. Modulation of p75NTR enhances trophic support by decreasing the levels of proNGF and proBDNF and increasing the mature form of NGF and BDNF. The findings show that NGF enhanced angiogenesis and tube formation of MSCs in PI3K/Akt-dependent manner. Additionally, NGF was shown to promote endothelial progenitor cell mediated angiogenic responses via SDF-1/CXCR-4, with minimal effect on resident retinal endothelial cells.

Of note, changes in the gene expression of both survival and apoptotic markers were explored in HREs upon treatment with the CM of p75NTR-silenced MSCs. Conventionally, the Bcl-2/Bax axis regulates mitochondrial-induced apoptosis in endothelial cells through the reciprocation of the anti-apoptotic proteins; Bcl-2 and the pro-apoptotic protein; Bax, which might result in a neutral net effect. Remarkably, marked increases in the expression of both Bcl-2 and Bax in HREs treated with CM of p75NTR-silenced MSCs was observed. Although the pro-apoptotic marker BAX was increased, other pro-apoptotic markers, such as p53 and caspase-3 transcripts, did not show significant difference from the controls (FIG. 5). In light of other increased survival and angiogenic factors, such as VEGF and NGF, it is conceivable that the CM of p75NTR-silenced MSCs exerted an anti-apoptotic and proangiogenic effect on HREs.

In parallel to genetic silencing of MSCs-p75NTR data, pharmacological inhibition of MSCs-p75NTR using LM11A-31 enriched MSCs secretome with SDF-1α, NGF and VEGFA and augmented the paracrine angiogenic behavior on endothelial cells in vitro (FIG. 7 and FIG. 8). LM11A-31 (2-amino-3-methyl-pentanoic acid [2-morpholin-4-yl-ethyl]-amide) is a water-soluble isoleucine derivative. The specificity of LM11A-31 to p75NTR has been documented by the complete loss of its protective activity in cultures of p75NTR−/− neurons. It has been demonstrated that the anti-inflammatory and neuro- and vascular protective effects of LM11A-31. However, this is the first report showing the impact of LM11A-31 on stem cells and its protective effects on visual acuity in a mouse model of the ischemic retina (FIG. 9). At the dose used (50 mg/kg), LM11A-31 has been shown to cross the blood-brain barrier following oral administration. Of note, LM11A-31 was approved by the US Food and Drug Administration as an investigational new drug, successfully completed a phase I safety trial in normal young and elderly individuals and is currently undergoing evaluation in a phase 2a exploratory endpoint trial in Alzheimer's disease (ClinTrials.gov registration no. NCT03069014).

Among stem cells including embryonic, neural and adult stem cells, MSCs gained interest because they can be obtained from the patients' bone marrow in quantities appropriate for clinical application. Since the expansion of MSCs from each patient can be possibly hampered by the disease state, allogenic cell therapy will be the way to go. Here, evidence is provided that combination of p75NTR modulation strategy and MSCs injection can serve as novel therapeutic approach to rescue visual function in ischemic retinal diseases with superior therapeutic potential than solely using MSCs. The underlying mechanism can involve augmented SDF-1, VEGF and NGF signaling pathways. The current findings support the therapeutic benefit of the orally bioavailable compound, LM11A-31 for ischemic retinal diseases. While homing and integration of MSCs into retina vasculature were observed, that process was correlated with vascular protection post-ischemic injury.

### 4. Materials and Methods

### i. Animals

All animal experiments were conducted in agreement with Association for Research in Vision and Ophthalmology statement for use of animals in ophthalmic and vision research, and Charlie Norwood VA Medical Center Animal Care and Use Committee (ACORP #16-01-088). The p75NTR, B6.129S4Ngfrtm1Jae (p75NTR−/−, exon III knockout mice were obtained from Jackson Laboratories (Bar Harbor, Maine, USA) and crossed with C57BL6-J mice (Jackson Laboratories, Bar Harbor, Maine, USA). These mice were crossed and back-crossed to establish a colony of homozygous p75NTR−/− and WT breeders that produced the mice used in the current study.

### ii. Retinal Ischemia/Reperfusion

For surgeries, mice were anesthetized with intraperitoneal ketamine (50 mg/kg; Hospira, Inc., Lake Forest, IL, USA) and xylazine (10 mg/kg; Akorn, Decatur, IL, USA). Retinal ischemia/reperfusion (I/R) was performed as described previously in Coucha, M. et al. Modulating Expression of Thioredoxin Interacting Protein (TXNIP) Prevents Secondary Damage and Preserves Visual Function in a Mouse Model of Ischemia/Reperfusion. Int. J. Mol. Sci. 2019, 20. Briefly, pupils were dilated with 1% Atropine Sulfate (Akorn, Inc., Lake Forest, IL, USA). The anterior chamber was cannulated with a 32 gauge needle attached to a line from a saline reservoir at a height calibrated to yield 120 mmHg. The intraocular pressure (IOP) was elevated to 120 mmHg for 45-60 min. I/R injury and choroidal nonperfusion was evident by whitening of the anterior segment of the globe and blanching of the episcleral veins. During infusion, topical anesthesia (0.5% tetracaine HCL, Bausch & Lomb, NJ, USA) was applied to the cornea. After ischemia, the needle was immediately withdrawn, allowing for rapid reperfusion, IOP was normalized, and reflow of the retinal vasculature was confirmed by observation of the episcleral veins. Topical antibiotic (NeoPolycin, Perrigo, Allegan, MI, USA) was applied to the cornea to minimize infection. I/R injury was performed in one eye with the other undergoing sham surgery, in which the needle was inserted into the anterior chamber without elevating the IOP. Mice were sacrificed 10 days post I/R and eyes were processed.

### iii. Mesenchymal Stem Cells (MSCs) Culture

GFP-labeled mouse MSCs were obtained as a kind gift from Dr. William D. Hill (MUSC, Charleston, SC) [57] and used between passages 5-9. MSCs cells were cultured in high glucose DMEM (4.5 g/L D-glucose, Thermo-Fisher Grand Island, NY, USA) supplemented with 10% FBS and 1% penicillin/streptomycin at 37° C. and 5% CO2. Knocking down p75NTR expression was performed according to manufacturer's instructions (Santa Cruz Biotechnology Inc., Dallas, TX, USA). Briefly, MSCs were shifted to antibiotic-free medium over night when 60-80% confluent. Cells were transfected with Si-RNA against p75NTR receptor (sc-37268) or scrambled (sc-37007) with the aid of lipofectamine transfection reagent (sc-29528) for 6 h. DMEM medium containing 2× of FBS and antibiotic was added on top of pure transfection medium for an extra 12 h. Next, transfection medium was removed and cells were allowed to recover in 1×DMEM medium for 6 h. For in vivo studies, cells were collected in sterile PBS (50,000 cells/μL) for intravitreal injection 48 h after I/R. For additional set of experiments, MSCs were treated with the p75NTR modulator (LM11A-31, 200 nM). Confluent MSCs (80% confluent) were treated with LM11A-31 or its vehicle for 12 h. Then, condition media were collected as well as cell lysates using mirVANA™ PARIS™ Kit (Ambion Inc., Austin, TX, USA), according to manufacturer's instructions.

### iv. Intravitreal Injection of MSCs

Mice were anesthetized by intraperitoneal injection of ketamine (50 mg/kg, Hospira, Inc., Lake Forest, IL, USA) and xylazine (10 mg/kg, Akorn, Decatur, IL, USA) mixture and complete anesthesia was confirmed by loss of reflex to sharp paw pinch. MSCs (100,000 cells/2 μL sterile PBS/eye) were injected intravitreally 48 h after I/R using a Hamilton syringe with a 33 gauge glass capillary.

### v. Vascular Localization of MSCs

3 or 7 days post intravitreal injection of MSCs, mice were euthanized in CO2 chamber (2% flow rate for 5 min), followed by cervical dislocation. Eyes were enucleated and fixed in 2% paraformaldehyde overnight. Retinas were dissected and permeabilized for 15 min with 0.3% Triton X-PBS then stained overnight at 4° C. with isolectin B4; biotinylated griffonia (bandeiraea) simplicifolia lectin I (GSL I, BSL I), (Vector Labs, Burlingame, CA, USA; 1% in 5% normal goat serum in 0.3% Triton X-PBS), followed by incubation with secondary antibody; Texas red® avidin D (Vector labs, Burlingame, CA, USA; 0.5% in 5% normal goat serum in 0.3% Triton X-PBS). Lectin-stained retinas were flat mounted onto Super-frost/Plus microscope slides (Fisher Scientific, Waltham, MA, USA) with the photoreceptor side facing down and imbedded in Vectashield mounting media for fluorescence (Vector Labs, Burlingame, CA, USA). Slides were photomicrographed at 40× using a Zeiss Axio Observer Z1 (Carl Zeiss Vision Inc., Thornwood, NY, USA).

### vi. Isolation of Retinal Vasculature and Determination of Aacellular Capillaries

The retinal vasculature was isolated as described previously [58]. Freshly enucleated eyes were fixed with 2% paraformaldehyde overnight. Retinal cups were dissected, washed in PBS, then incubated with 3% Difco-trypsin 250 (BD Biosciences, San Jose, CA, USA) in 25 mmol/l Tris buffer, pH 8, at 37° C. for 1-1.5 h. Vitreous and nonvascular cells were gently removed from the vasculature, which was soaked in several washes of 0.5% Triton X-100 to remove the neuronal retina. Trypsin-digested retinas were stained with periodic acid-Schiff and hematoxylin (PASH). Numbers of acellular capillaries were quantified in six different areas of the mid-retina using bright field microscopy (20×) in a masked manner by two different researchers. Acellular capillaries were identified as capillary-sized blood vessel tubes with no nuclei along their length.

### vii. Endothelial Cell Cultures

All human retinal endothelial (HRE) cell studies were in accordance with Association for Research in Vision and Ophthalmology and Charlie Norwood Veterans Affairs Medical Center, research and ethics committee. HREs and supplies were purchased from Cell Systems Corporation (Kirkland, WA, USA) and VEC Technology Inc. (Rensselaer, NY, USA).

### viii. Real-Time Quantitative PCR

Retinas samples and MSCs lysates were processed using (mirVANA™ PARIS™ Kit, Ambion Inc., Austin, TX, USA) and RNA was purified and quantified as described by the manufacturer's instructions. A one-step quantitative RT-PCR kit (Invitrogen, Carlsbad, CA, USA) was used to amplify 10 ng retinal mRNA. Quantitative PCR was conducted using a StepOnePlus qPCR system (Applied BioSystems, Life Technologies, Waltham, MA, USA). For HREs, RNA isolation was performed using the TRIzol Reagent (Invitrogen, Waltham, MA, USA), according to the manufacturer's instructions. RNA concentration was quantified by spectrophotometry at 260 nm using Nanodrop 2000 (Thermo Fisher Scientific, Waltham, MA, USA). A total of 1 μg of RNA was reverse transcribed to prepare cDNA using the RNA to cDNA EcoDry premix (Takara Bio USA Inc., Mountain View, CA, USA). Real-time PCR was performed using the CFX96 PCR instrument with matched primers (See Table 1) and Universal SYBR Green Supermix (Bio-Rad, Hercules, CA, USA). The following PCR parameters were used: initial denaturation cycle at 95° C. for 3 min, followed by 40 amplification cycles at 95° C. for 10 s, 56° C. for 15 s, and 72° C. for 1 min. The results are presented as the fold change in relative gene expression quantified using the delta-delta CT method and normalized to internal controls (18 S, GAPDH) and expressed relative to controls.

TABLE 1

| Primer | Sequence (5′-3′) |
|---|---|
| Mouse p75^{NTR} F | CCTGCCTGGACAGTGTTACG (SEQ ID NO: 2) |
| Mouse p75^{NTR} R | CACACAGGGAGCGGACATAC (SEQ ID NO: 3) |
| Mouse VEGFA F | GTACCTCCACCATGCCAAGT (SEQ ID NO: 4) |
| Mouse VEGFA R | GCATTCACATCTGCTGTGCT (SEQ ID NO: 5) |
| Mouse Akt1 F | ATGAACGACGTAGCCATTGTG (SEQ ID NO: 6) |
| Mouse Akt1 R | TTGTAGCCAATAAAGGTGCCAT (SEQ ID NO: 7) |
| Mouse Bcl-2 F | GTGGTGGAGGAACTCTTCA (SEQ ID NO: 8) |
| Mouse Bcl-2 R | GTTCCACAAAGGCATCCCAG (SEQ ID NO: 9) |
| Mouse Bax F | AGCAAACTGGTGCTCAAGGC (SEQ ID NO: 10) |
| Mouse Bax R | CCACAAAGATGGTCACTGTC (SEQ ID NO: 11) |
| Mouse Caspase-3 F | CCTCAGAGAGACATTCATGG (SEQ ID NO: 12) |
| Mouse Caspase-3 R | GCAGTAGTCGCCTCTGAAGA (SEQ ID NO: 13) |
| Mouse P53 F | GGAAATTTGTATCCCGAGTATCTG (SEQ ID NO: 14) |
| Mouse P53 R | GTCTTCCAGTGTGATGATGGTAA (SEQ ID NO: 15) |
| Mouse GAPDH F | CACATTGGGGGTAGGAACAC (SEQ ID NO: 16) |
| Mouse GAPDH R | AACTTTGGGCATTGTGGAAGG (SEQ ID NO: 17) |

### ix. Cell Migration Assay

HREs were grown to confluence and then were wound with a single sterile cell scraper of constant diameter. Cells were divided into three groups, treated with regular media without FBS, and 1:1 mixture of FBS-free regular media and CM of vehicle- or treated-MSCs. Images of wounded areas were taken immediately after adding the treatment using a phase contrast microscope, and baseline were marked under the cell culture dish with blue marker. After 12 h, images of same field were taken and % cell migration was calculated. Each condition was verified in triplicate and was repeated using independent cultures.

### x. Tube Formation Assay

Corning Matrigel Matrix (10 mg protein/mL) Growth Factor Reduced (Corning Inc., Cat. No. 354230, Corning, NY, USA) was used for tube formation assay. Then, 289 μL of cold (4° C.) Matrigel per well was added into chilled 24-well flat-bottom tissue culture plate (USA Scientific Inc., Ocala, FL, USA) and polymerized for 1 h at 37° C., 5% CO2 incubator. HREs were trypsinized and separated into different groups. Each group of cells was centrifuged and cell pellets were resuspended in regular media without FBS, same media with 1:1 mixture of FBS free regular media and CM of vehicle- or treated-MSCs. Then 300 μL of the cell suspension was added onto each well of Matrigel coated 24-well culture plate and incubated at 37° C. After 24-h, cells forming tube structures were analyzed by microscopy. Cells in five replicate fields of triplicate wells were digitally photographed with an EVOS microscope (Thermo Fisher Scientific Inc., Waltham, MA, USA). The images for Tube formation assay were analyzed using Angiogenesis analyzer for imageJ. An area of 1.8 mm2 from each well was analyzed with imageJ ver. 1.53c, and 3 wells were analyzed per group. The software automatically generates the skeletonized trees overlay to highlight junction, branches, and segments (FIG. 6C lower panel, FIG. 8C lower panel). Master junctions were identified as junctions linking at least three master segments. The total tubular length was calculated as the average of the total tubule length from three wells, five random fields per well.

### xi. Visual Acuity Test

Visual acuity was assessed behaviorally by training and testing mice on the “cue” version of the Morris water maze task. Mice used for this experiment underwent I/R in both eyes. They were trained for 3 days before I/R and visual acuity was tested at 3, 6, 9, 12 and 15 days post I/R. Animals were placed for 10 s on a platform in a tank of opaque water, 22-25° C., which was elevated above the water surface (2 cm) and clearly visible from any location in the tank. Subsequently, there were four trials per day for 3 days or until a stable performance plateau was reached. On each trial, animals started from different locations at the periphery of the tank and were allowed to swim to the escape platform. If they did not reach the platform in 60 s, they were gently guided to it by the investigator. They remained on the platform for 10 s. Visual impairment was diagnosed by higher escape time. Treatment groups received oral gavage of LM11A-31 (50 mg/kg/day) every other day, starting 48 h post I/R.

### xii. Statistical Analysis

All the data are expressed as mean±SEM. Differences between 2 groups were detected using unpaired Student T-test. One-way ANOVA was used to assess significant differences between 3 groups. Two-way ANOVA was used to assess interaction between two variables: 2 genes (WT vs. p75NTR−/−) X I/R exposure (I/R vs Sham). Tukey-Kramer post-multiple comparisons was used for significant interactions among various groups. Significance for all tests was determined at α=0.05, Graphpad Prism, Ver.6.

### xiii. Abbreviations

Akt: Protein Kinase B; Bax: Bcl-2-like protein 4; Bcl-2B-cell lymphoma 2; CM: Conditioned medium; CXCR-4: Chemokine receptor type 4; CXCR-7: Chemokine receptor type 7; HREs Human retinal endothelial cells; I/R Ischemia reperfusion; MSCs Mesenchymal stem cells; NGF: Nerve growth factor; PASH: Periodic acid-Schiff and hematoxylin; P75NTR: p75 neurotrophin receptor; PDR: Proliferative diabetic retinopathy; SDF-1: Stromal cell-derived factor-1; VEGF: Vascular endothelial growth factor

### B. Example 2: Effects of LM11A-31 on Diabetes

Diabetes affects the whole body by causing a state of mild inflammation that can slow down the rolling of white blood cells to block or damage the small capillaries especially in the eye, kidney and the limbs. Leakage or death of the small capillaries leads to accumulation of fluids and limits the ability to regenerate after exposure to ischemic limb. Leakage of fluids in front of the macula disturb in visual acuity and results in appearance of protein the urine of diabetic patient. Current therapeutic options are limited for early stages they do not treat the whole body. Therefore, there is a great need to identify novel therapeutic targets that can improve the inflammation of the whole body and preserve both capillaries and neurons in the eye, kidney and the limbs. Translational studies showed the benefit of a new drug, LM11A-31 that can prevent the inflammation in the whole body and in particular the eye and kidney after the onset of diabetes. The studies quantified the benefit of the new drug on improving the damage of the small capillaries and fluid accumulation especially in the eye as well as visual function in ischemic and diabetic animals.

Diabetes and its microvascular complications are perceived as systemic low-grade inflammation manifested by positive association of circulating markers of inflammation. Nevertheless, several systemic orally administered drugs failed repeatedly in clinical trials. Hence, there is a need to identify viable targets that are “upstream” of the terminal effectors involved in the disease. The efficacy of a small p75^{NTR} inhibitor, an orally bioavailable and safe drug (LM11A-31), has been used in an intervention using clinically relevant endpoints. The studies provide strong rationale for systemic targeting of the proinflammatory action of p75^{NTR} and that it has proven efficacious in preventing diabetes-associated microvascular injury in retina, kidney and ischemic limbs.

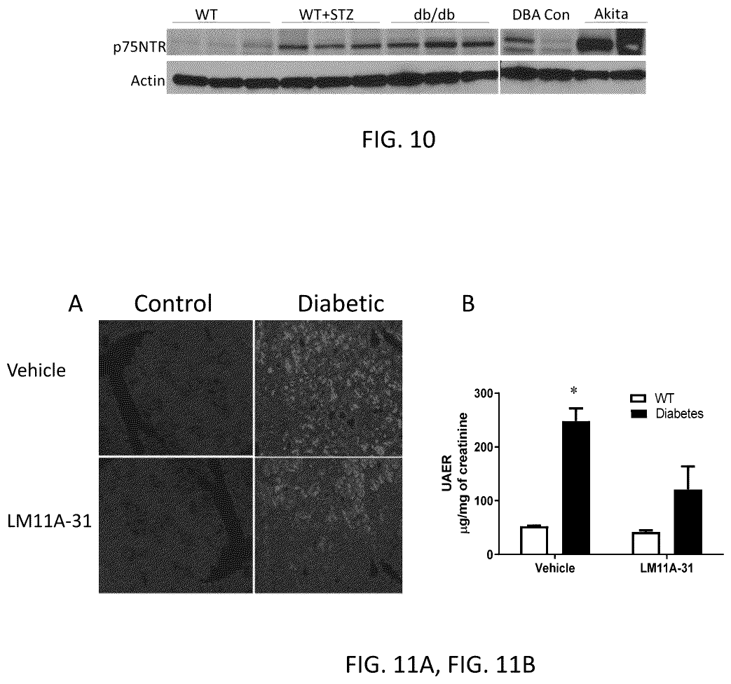

FIG. 10 shows that diabetes significantly increases expression of p75^{NTR} in the kidney. Significant increases of p75^{NTR} expression have been shown in ocular samples from patients with Type-1 diabetes (insulin dependent) and Type-2 diabetes (non-insulin dependent). Here, the impact of various types of diabetes on renal expression of p75^{NTR} is shown. For Type-1 diabetes, streptozotocin (STZ), a chemical model to deplete insulin, was used and male Akita mice, a genetic model that lack expression of insulin. For Type-2 diabetes, db/db mice, a genetic model that show insulin resistance, obesity and hyperglycemia, were used. Western blot analysis from kidney samples from these various models showed significant increases in p75^{NTR} expression when compared to their corresponding controls. Both Akita mice and STZ (Type-1 diabetes)) were compared to controls (WT) and db/db mouse (Type-2) was compared to its DBA control. These results show that both types of chronic diabetes (16-weeks) universally increase p75^{NTR} in the kidney.

FIG. 11A shows immunohistochemistry of kidney sections from STZ-mice (model for Type-1 diabetes) showing marked increases in expression of p75^{NTR} after 16-weeks when compared to controls. Interventional treatment with the p75^{NTR} modulator LM11A-31 (50 mg/Kg, po, QOD) for 12 weeks did not alter p75^{NTR} expression in diabetic or control animals. FIG. 11B shows the impact of diabetes and modulating p75^{NTR} on blood glucose level. Treatment with LM11A-31 (50 mg/Kg, po, QOD) for 12 weeks did not alter blood glucose level in control or diabetic animals. FIG. 11C shows the impact of diabetes and modulating p75^{NTR} on body weight. Diabetes significantly reduced the weight when compared to controls (vehicle-treated animals). Interestingly, treatment with LM11A-31 (50 mg/Kg, po, QOD) for 12 weeks preserved the weight of diabetic animals compared to controls (* P<0.05 versus the rest of the groups, n=6-7).

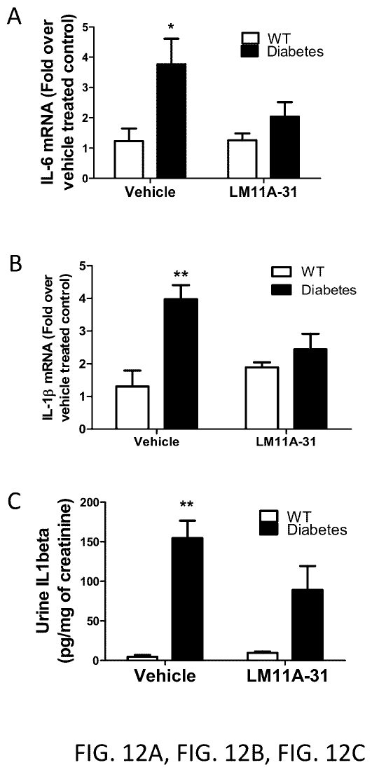

FIG. 12 shows diabetic nephropathy progresses from microalbuminuria to macroalbuminuria, renal failure and end stage of renal disease. Currently, there are no effective therapies available to prevent the development of diabetic nephropathy. Thus, early diagnosis of microalbuminuria using laboratory biomarkers can facilitate early treatment of diabetic nephropathy. At 24-h urine collection for urine albumin measurement is the gold standard method for screening microalbuminuria. Urinary albumin excretion rate (UAER) normalized to creatinine is the standard biomarker to diagnose renal injury and stage diabetic nephropathy. FIG. 12A shows that diabetes (16-weeks) caused significant renal injury evident by 5-fold increases in UAER normalized to creatinine. Interventional treatment with LM11A-31 (50 mg/Kg, po, QOD) for 12 weeks significantly improved UAER in diabetic animals and did not alter baseline in control. FIG. 12B shows that diabetes (16-weeks) caused significant increase (1.4-fold) in the ratio of kidney weight to body weight compared to controls. Ratio of kidney to body weight has been positively correlated to glomeruli number and size. Interventional treatment with LM11A-31 (50 mg/Kg, po, QOD) for 12 weeks significantly improved the ratio of kidney weight to body weight in diabetic animals and did not alter the baseline in treated controls.

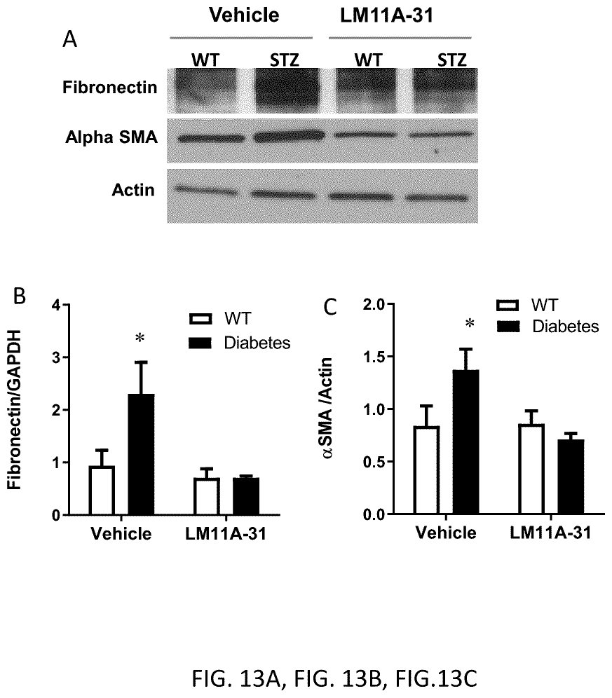

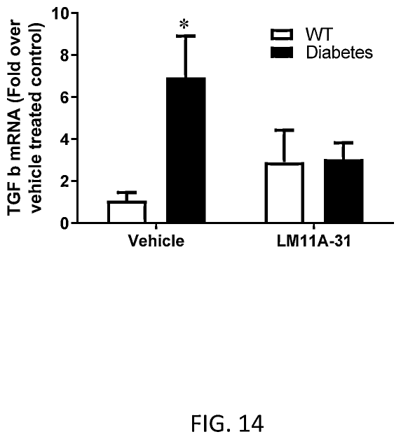

FIGS. 13 and 14 show impact of diabetes and modulating p75^{NTR} on kidney fibrosis. Immunohistological assessment of fibrotic changes using Trichome staining showed that diabetes markedly increased renal fibrosis. Trichrome stains the muscle tissue (red), and the collagen fibers (blue). Diabetes increased collagen deposition (Blue) and number of dilated glomeruli compared to controls (FIG. 13). Interventional treatment with LM11A-31 (50 mg/Kg, po, QOD) for 12 weeks significantly improved fibrosis and preserved glomeruli size. Histological staining with periodic acid-schiff (PAS) highlight basement membranes of glomerular capillary loops and tubular epithelium. Results showed that diabetes markedly increased mesangial matrix expansion in glomeruli compared to controls that show normal size (FIG. 14). Interventional treatment with LM11A-31 (50 mg/Kg, po, QOD) for 12 weeks significantly preserved glomeruli areas back to normal glomeruli size.

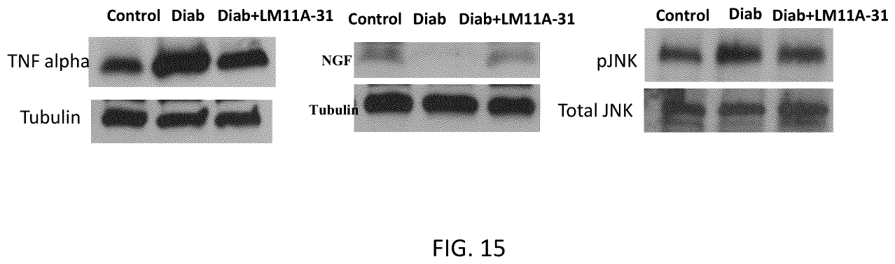

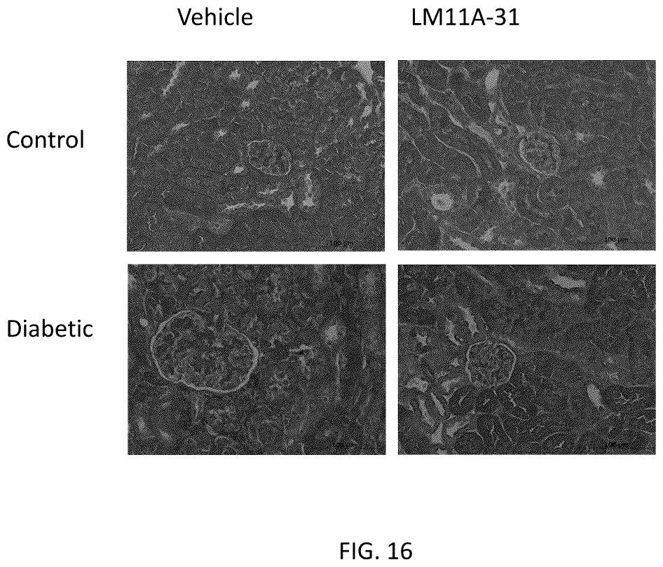

FIGS. 15 and 16 show impact of diabetes and modulating p75^{NTR} on TGF-beta and fibrosis markers. Chronic diabetes is known to increase the expression of pro-fibrotic cytokines (TNFα and TGF-β1). TGFβ induces epithelial to mesenchymal transition of tubular epithelial cells into myofibroblasts and considered as principal mechanism of renal fibrosis. Results showed that diabetes significantly increased expression of TGF-beta (7-fold) when compared to controls (FIG. 15). Interventional treatment with LM11A-31 (50 mg/Kg, po, QOD) for 12 weeks significantly prevented the increase in TGF-beta expression. During kidney injury, pericytes promptly migrate away from the capillary wall into the interstitial space differentiated into myofibroblast and contribution to kidney fibrosis. Next, we examined expression of myofibroblast markers. Consistent with Masson's trichrome and immunostaining, Western Blot analysis showed that diabetes significantly increased expression of fibrosis markers; 2.5-fold increase in Fibronectin and 1.65-fold in alpha-SMA expression compared to controls. Interventional treatment with LM11A-31 (50 mg/Kg, po, QOD) for 12 weeks significantly prevented the increase in fibrotic markers in diabetic animals but it did not alter base-line in controls.

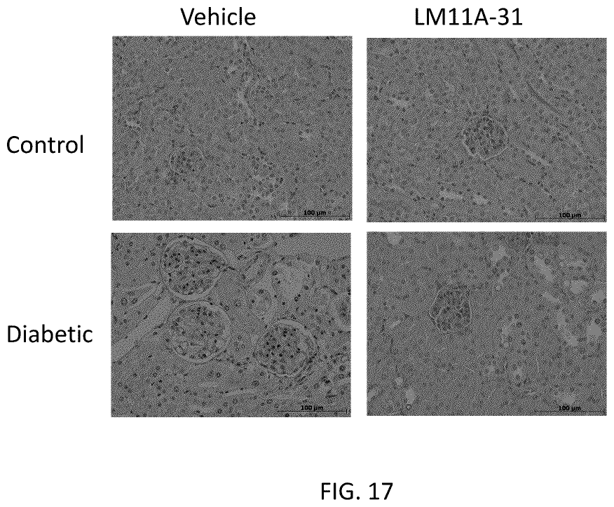

FIGS. 17 and 18 show impact of diabetes and modulating p75^{NTR} on kidney inflammatory markers. Prior work demonstrated the significance of elevated IL-6 expression in diabetic tubular epithelial cells. Here, we checked expression of inflammatory markers mRNA and results showed ˜4-fold increase of IL-6 mRNA and IL-1β expression in kidney compared to controls (FIG. 17). Urine level of IL-1β assessed by ELISA was very low in vehicle-treated controls, however, diabetes triggered sharp increase in IL-1β expression. Interventional treatment with LM11A-31 (50 mg/Kg, po, QOD) for 12 weeks significantly prevented the increase in renal and urinary inflammation in diabetes. FIG. 18 Western Blot analysis shows diabetes (16 weeks) significantly increases expression of inflammatory markers TNF-α, and apoptotic marker p-JNK (n=3) compared to control. Similar to our prior observation in the diabetic retina, diabetes caused significant decreases in the nerve growth factor (NGF) in kidney compared to controls. NGF is a survival and anti-apoptotic neurotrophin. Interventional treatment with LM11A-31 (50 mg/Kg, po, QOD) for 12 weeks significantly prevented the increase in TNF-a, apoptotic marker p-JNK and as well as restored NGF levels in diabetes. Interestingly, modulating p75^{NTR} in diabetes has been shown to shift the balance from inflammatory to restoring endogenous trophic support.

Those skilled in the art will recognize, or be able to ascertain using no more than routine experimentation, many equivalents to the specific embodiments of the method and compositions described herein. Such equivalents are intended to be encompassed by the following claims.

### REFERENCES

1. Das, A.; McGuire, P. G. Retinal and choroidal angiogenesis: Pathophysiology and strategies for inhibition. Prog. Retin. Eye Res. 2003, 22, 721-748.
2. Mozaffarieh, M.; Grieshaber, M. C.; Flammer, J. Oxygen and blood flow: Players in the pathogenesis of glaucoma. Mol. Vis. 2008, 14, 224-233.
3. Coucha, M.; Elshaer, S. L.; Eldahshan, W. S.; Mysona, B. A.; El-Remessy, A. B. Molecular mechanisms of diabetic retinopathy: Potential therapeutic targets. Middle East. Afr J. Ophthalmol. 2015, 22, 135-144, doi:10.4103/0974-9233.154386.
4. Arevalo, J. F.; Liu, T. Y. A. Pan-American Collaborative Retina Study, G. Intravitreal Bevacizumab in Diabetic Retinopathy. Recommendations from the Pan-American Collaborative Retina Study Group (PACORES): The 2016 Knobloch Lecture. Asia Pac. J. Ophthalmol. (Philo) 2018, 7, 36-39, doi:10.22608/AP0.2017466.
5. Ding, S. L. S.; Kumar, S.; Mok, P. L. Cellular Reparative Mechanisms of Mesenchymal Stem Cells for Retinal Diseases. Int. J. Mol. Sci. 2017, 18, doi:10.3390/ijms18081406.
6. Berglund, A. K.; Fortier, L. A.; Antczak, D. F.; Schnabel, L. V. Immunoprivileged no more: Measuring the immunogenicity of allogeneic adult mesenchymal stem cells. Stem Cell Res. Ther. 2017, 8, 288, doi:10.1186/s13287-017-0742-8.
7. Le Blanc, K.; Tammik, C.; Rosendahl, K.; Zetterberg, E.; Ringden, O. HLA expression and immunologic properties of differentiated and undifferentiated mesenchymal stem cells. Exp. Hematol. 2003, 31, 890-896, doi:10.1016/s0301-472x(03)00110-3.
8. Lalu, M. M.; McIntyre, L.; Pugliese, C.; Fergusson, D.; Winston, B. W.; Marshall, J. C.; Granton, J.; Stewart, D. J.; Canadian Critical Care Trials, G. Safety of cell therapy with mesenchymal stromal cells (SafeCell): A systematic review and meta-analysis of clinical trials. PLoS ONE 2012, 7, e47559, doi:10.1371/journal.pone.0047559.
9. Meirelles Lda, S.; Fontes, A. M.; Covas, D. T.; Caplan, A. I. Mechanisms involved in the therapeutic properties of mesenchymal stem cells. Cytokine Growth Factor Rev. 2009, 20, 419-427, doi:10.1016/j.cytogfr.2009.10.002.
10. Elshaer, S. L.; Evans, W.; Pentecost, M.; Lenin, R.; Periasamy, R.; Jha, K. A.; Alli, S.; Gentry, J.; Thomas, S. M.; Sohl, N.; et al. Adipose stem cells and their paracrine factors are therapeutic for early retinal complications of diabetes in the Ins2(Akita) mouse. Stem Cell Res. Ther. 2018, 9, 322, doi:10.1186/s13287-018-1059-y.
11. Mazo, M.; Cemborain, A.; Gavira, J. J.; Abizanda, G.; Arana, M.; Casado, M.; Soriano, M.; Hernandez, S.; Moreno, C.; Ecay, M.; et al. Adipose stromal vascular fraction improves cardiac function in chronic myocardial infarction through differentiation and paracrine activity. Cell Transplant. 2012, 21, 1023-1037, doi:10.3727/096368911X623862.
12. Kicic, A.; Shen, W. Y.; Wilson, A. S.; Constable, U.; Robertson, T.; Rakoczy, P. E. Differentiation of marrow stromal cells into photoreceptors in the rat eye. J. Neurosci. 2003, 23, 7742-7749.
13. Labrador-Velandia, S.; Alonso-Alonso, M. L.; Di Lauro, S.; Garcia-Gutierrez, M. T.; Srivastava, G. K.; Pastor, J. C.; Fernandez-Bueno, I. Mesenchymal stem cells provide paracrine neuroprotective resources that delay degeneration of co-cultured organotypic neuroretinal cultures. Exp. Eye Res. 2019, 185, 107671, doi:10.1016/j.exer.2019.05.011.
14. Borkowska-Kuczkowska, A.; Slugocka, D.; Swiatkowska-Flis, B.; Boruczkowski, D. The use of mesenchymal stem cells for the treatment of progressive retinal diseases: A review. Regen. Med. 2019, 14, 321-329, doi:10.2217/rme-2019-0022.
15. Elshaer, S. L.; El-Remessy, A. B. Implication of the neurotrophin receptor p75(NTR) in vascular diseases: Beyond the eye. Expert Rev. Ophthalmol. 2017, 12, 149-158, doi: 10.1080/17469899.2017.1269602.
16. Tomellini, E.; Lagadec, C.; Polakowska, R.; Le Bourhis, X. Role of p75 neurotrophin receptor in stem cell biology: More than just a marker. Cell Mol. Life Sci. 2014, 71, 2467-2481, doi:10.1007/s00018-014-1564-9.
17. Jiang, J.; Lv, Z.; Gu, Y.; Li, J.; Xu, L.; Xu, W.; Lu, J.; Xu, J. Adult rat mesenchymal stem cells differentiate into neuronal-like phenotype and express a variety of neuro-regulatory molecules in vitro. Neurosci. Res. 2010, 66, 46-52, doi:10.1016/j.neures.2009.09.1711.
18. Mikami, Y.; Ishii, Y.; Watanabe, N.; Shirakawa, T.; Suzuki, S.; Irie, S.; Isokawa, K.; Honda, M. J. CD271/p75(NTR) inhibits the differentiation of mesenchymal stem cells into osteogenic, adipogenic, chondrogenic, and myogenic lineages. Stem Cells Dev. 2011, 20, 901-913, doi:10.1089/scd.2010.0299.
19. Mysona, B. A.; Al-Gayyar, M. M.; Matragoon, S.; Abdelsaid, M. A.; El-Azab, M. F.; Saragovi, H. U.; El-Remessy, A. B. Modulation of p75(NTR) prevents diabetes- and proNGF-induced retinal inflammation and blood-retina barrier breakdown in mice and rats. Diabetologia 2013, 56, 2329-2339, doi:10.1007/s00125-013-2998-6.
20. Shanab, A. Y.; Mysona, B. A.; Matragoon, S.; El-Remessy, A. B. Silencing p75(NTR) prevents proNGF-induced endothelial cell death and development of acellular capillaries in rat retina. Mol. Ther Methods Clin. Dev. 2015, 2, 15013, doi:10.1038/mtm.2015.13.
21. Mohamed, R.; Shanab, A. Y.; El Remessy, A. B. Deletion of the Neurotrophin Receptor p75(NTR) Prevents Diabetes-Induced Retinal Acellular Capillaries in Streptozotocin-Induced Mouse Diabetic Model. J. Diabetes Metab. Disord. Control 2017, 4, doi:10.15406/jdmdc.2017.04.00129.
22. Elshaer, S. L.; El-Remessy, A. B. Deletion of p75(NTR) prevents vaso-obliteration and retinal neovascularization via activation of Trk-A receptor in ischemic retinopathy model. Sci. Rep. 2018, 8, 12490, doi:10.1038/s41598-018-30029-0.
23. Barcelona, P. F.; Sitaras, N.; Galan, A.; Esquiva, G.; Jmaeff, S.; Jian, Y.; Sarunic, M. V.; Cuenca, N.; Sapieha, P.; Saragovi, H. U. p75NTR and Its Ligand ProNGF Activate Paracrine Mechanisms Etiological to the Vascular, Inflammatory, and Neurodegenerative Pathologies of Diabetic Retinopathy. J. Neurosci. 2016, 36, 8826-8841, doi:10.1523/JNEUROSCI.4278-15.2016.
24. Wei, Y.; Wang, N.; Lu, Q.; Zhang, N.; Zheng, D.; Li, J. Enhanced protein expressions of sortilin and p75NTR in retina of rat following elevated intraocular pressure-induced retinal ischemia. Neurosci. Lett. 2007, 429, 169-174, doi:10.1016/j.neulet.2007.10.012.
25. Lu, B.; Pang, P. T.; Woo, N. H. The yin and yang of neurotrophin action. Nat. Rev. Neurosci. 2005, 6, 603-614, doi:10.1038/nrn1726.
26. Nakamura, T.; Endo, K.; Kinoshita, S. Identification of human oral keratinocyte stem/progenitor cells by neurotrophin receptor p75 and the role of neurotrophin/p75 signaling. Stem Cells 2007, 25, 628-638, doi:10.1634/stemcells.2006-0494.
27. Gentry, J. J.; Casaccia-Bonnefil, P.; Carter, B. D. Nerve growth factor activation of nuclear factor kappaB through its p75 receptor is an anti-apoptotic signal in RN22 schwannoma cells. J. Biol. Chem. 2000, 275, 7558-7565, doi:10.1074/jbc.275.11.7558.
28. Coulson, E. J.; Reid, K.; Shipham, K. M.; Morley, S.; Kilpatrick, T. J.; Bartlett, P. F. The role of neurotransmission and the Chopper domain in p75 neurotrophin receptor death signaling. Prog. Brain Res. 2004, 146, 41-62, doi:10.1016/S0079-6123(03)46003-2.
29. Johnston, A. L.; Lun, X.; Rahn, J. J.; Liacini, A.; Wang, L.; Hamilton, M. G.; Parney, I. F.; Hempstead, B. L.; Robbins, S. M.; Forsyth, P. A.; et al. The p75 neurotrophin receptor is a central regulator of glioma invasion. PLoS Biol. 2007, 5, e212, doi:10.1371/journal.pbio.0050212.
30. Elshaer, S. L.; Alwhaibi, A.; Mohamed, R.; Lemtalsi, T.; Coucha, M.; Longo, F. M.; El-Remessy, A. B. Modulation of the p75 neurotrophin receptor using LM11A-31 prevents diabetes-induced retinal vascular permeability in mice via inhibition of inflammation and the RhoA kinase pathway. Diabetologia 2019, 62, 1488-1500, doi:10.1007/s00125-019-4885-2.
31. Stitt, A. W.; O'Neill, C. L.; O'Doherty, M. T.; Archer, D. B.; Gardiner, T. A.; Medina, R. J. Vascular stem cells and ischaemic retinopathies. Prog. Retin. Eye Res. 2011, 30, 149-166, doi: 10.1016/j.preteyeres.2011.02.001.
32. Quirici, N.; Soligo, D.; Bossolasco, P.; Servida, F.; Lumini, C.; Deliliers, G. L. Isolation of bone marrow mesenchymal stem cells by anti-nerve growth factor receptor antibodies. Exp. Hematol. 2002, 30, 783-791, doi:10.1016/s0301-472x(02)00812-3.
33. Arnhold, S.; Absenger, Y.; Klein, H.; Addicks, K.; Schraermeyer, U. Transplantation of bone marrow-derived mesenchymal stem cells rescue photoreceptor cells in the dystrophic retina of the rhodopsin knockout mouse. Graefes Arch. Clin. Exp. Ophthalmol. 2007, 245, 414-422, doi: 10.1007/s00417 -006-0382-7.
34. Li, N.; Li, X. R.; Yuan, J. Q. Effects of bone-marrow mesenchymal stem cells transplanted into vitreous cavity of rat injured by ischemia/reperfusion. Graefes Arch. Clin. Exp. Ophthalmol. 2009, 247, 503-514, doi:10.1007/s00417-008-1009-y.
35. Kaminski, N.; Koster, C.; Mouloud, Y.; Borger, V.; Felderhoff-Muser, U.; Bendix, I.; Giebel, B.; Herz, J. Mesenchymal Stromal Cell-Derived Extracellular Vesicles Reduce Neuroinflammation, Promote Neural Cell Proliferation and Improve Oligodendrocyte Maturation in Neonatal Hypoxic-Ischemic Brain Injury. Front. Cell Neurosci. 2020, 14, 601176, doi:10.3389/fnce1.2020.601176.
36. Huber, B.; Volz, A. C.; Kluger, P. J. How do culture media influence in vitro perivascular cell behavior? Cell Biol. Int. 2015, 39, 1395-1407, doi:10.1002/cbin.10515.
37. Xu, J.; Gong, T.; Heng, B. C.; Zhang, C. F. A systematic review: Differentiation of stem cells into functional pericytes. FASEB J. 2017, 31, 1775-1786, doi:10.1096/fj.201600951RRR.
38. Xu, L.; Li, J.; Luo, Z.; Wu, Q.; Fan, W.; Yao, X.; Li, Q.; Yan, H.; Wang, J. Abeta inhibits mesenchymal stem cell-pericyte transition through MAPK pathway. Acta Biochim. Biophys. Sin. (Shanghai) 2018, 50, 776-781, doi:10.1093/abbs/gmy072.
39. Urbich, C.; Aicher, A.; Heeschen, C.; Dernbach, E.; Hofmann, W. K.; Zeiher, A. M.; Dimmeler, S. Soluble factors released by endothelial progenitor cells promote migration of endothelial cells and cardiac resident progenitor cells. J. Mol. Cell Cardiol. 2005, 39, 733-742, doi:10.1016/j.yjmcc.2005.07.003.
40. Tang, J.; Wang, J.; Yang, J.; Kong, X.; Zheng, F.; Guo, L.; Zhang, L.; Huang, Y. Mesenchymal stem cells over-expressing SDF-1 promote angiogenesis and improve heart function in experimental myocardial infarction in rats. Eur. J. Cardiothorac Surg. 2009, 36, 644-650, doi: 10.1016/j.ejcts.2009.04.052.
41. Reiter, J.; Drummond, S.; Sammour, I.; Huang, J.; Florea, V.; Dornas, P.; Hare, J. M.; Rodrigues, C. O.; Young, K. C. Stromal derived factor-1 mediates the lung regenerative effects of mesenchymal stem cells in a rodent model of bronchopulmonary dysplasia. Respir. Res. 2017, 18, 137, doi:10.1186/s12931-017-0620-z.
42. Marquez-Curtis, L. A.; Janowska-Wieczorek, A. Enhancing the migration ability of mesenchymal stromal cells by targeting the SDF-1/CXCR4 axis. Biomed. Res. Int. 2013, 2013, 561098, doi:10.1155/2013/561098.
43. Chen, L.; Li, Y.; Chen, W.; Han, N.; Li, K.; Guo, R.; Liu, Z.; Xiao, Y. Enhanced recruitment and hematopoietic reconstitution of bone marrow-derived mesenchymal stem cells in bone marrow failure by the SDF-1/CXCR4. J. Tissue Eng. Regen Med. 2020, 14, 1250-1260, doi:10.1002/term.3096.
44. Wei, S. T.; Huang, Y. C.; Hsieh, M. L.; Lin, Y. J.; Shyu, W. C.; Chen, H. C.; Hsieh, C. H. Atypical chemokine receptor ACKR3/CXCR7 controls postnatal vasculogenesis and arterial specification by mesenchymal stem cells via Notch signaling. Cell Death Dis. 2020, 11, 307, doi:10.1038/s41419-020-2512-2.
45. Wang, Y.; Fu, W.; Zhang, S.; He, X.; Liu, Z.; Gao, D.; Xu, T. CXCR-7 receptor promotes SDF-1alpha-induced migration of bone marrow mesenchymal stem cells in the transient cerebral ischemia/reperfusion rat hippocampus. Brain Res. 2014, 1575, 78-86, doi:10.1016/j.brainres.2014.05.035.
46. Abhinand, C. S.; Raju, R.; Soumya, S. J.; Arya, P. S.; Sudhakaran, P. R. VEGF-A/VEGFR2 signaling network in endothelial cells relevant to angiogenesis. J. Cell Commun. Signal. 2016, 10, 347-354, doi:10.1007/s12079-016-0352-8.
47. Ge, Q.; Zhang, H.; Hou, J.; Wan, L.; Cheng, W.; Wang, X.; Dong, D.; Chen, C.; Xia, J.; Guo, J.; et al. VEGF secreted by mesenchymal stem cells mediates the differentiation of endothelial progenitor cells into endothelial cells via paracrine mechanisms. Mol. Med. Rep. 2018, 17, 1667-1675, doi:10.3892/mmr.2017.8059.
48. Mead, B.; Chamling, X.; Zack, D. J.; Ahmed, Z.; Tomarev, S. TNFalpha-Mediated Priming of Mesenchymal Stem Cells Enhances Their Neuroprotective Effect on Retinal Ganglion Cells. Investig. Ophthalmol Vis. Sci. 2020, 61, 6, doi:10.1167/iovs.61.2.6.
49. Speidell, A.; Asuni, G. P.; Avdoshina, V.; Scognamiglio, S.; Forcelli, P.; Mocchetti, I. Reversal of Cognitive Impairment in gp120 Transgenic Mice by the Removal of the p75 Neurotrophin Receptor. Front. Cell Neurosci. 2019, 13, 398, doi:10.3389/fnce1.2019.00398.
50. Wang, W. X.; Hu, X. Y.; Xie, X. J.; Liu, X. B.; Wu, R. R.; Wang, Y. P.; Gao, F.; Wang, J. A. Nerve growth factor induces cord formation of mesenchymal stem cell by promoting proliferation and activating the PI3K/Akt signaling pathway. Acta Pharmacol. Sin. 2011, 32, 1483-1490, doi:10.1038/aps.2011.141.
51. Jadhao, C. S.; Bhatwadekar, A. D.; Jiang, Y.; Boulton, M. E.; Steinle, J. J.; Grant, M. B. Nerve growth factor promotes endothelial progenitor cell-mediated angiogenic responses. Investig. Ophthalmol. Vis. Sci. 2012, 53, 2030-2037, doi:10.1167/iovs.11-8430.
52. Molostvov, G.; Morris, A.; Rose, P.; Basu, S. Modulation of Bcl-2 family proteins in primary endothelial cells during apoptosis. Pathophysiol. Haemost. Thromb. 2002, 32, 85-91, doi:10.1159/000065081.
53. Tep, C.; Lim, T. H.; Ko, P. O.; Getahun, S.; Ryu, J. C.; Goettl, V. M.; Massa, S. M.; Basso, M.; Longo, F. M.; Yoon, S. O. Oral administration of a small molecule targeted to block proNGF binding to p75 promotes myelin sparing and functional recovery after spinal cord injury. J. Neurosci. 2013, 33, 397-410, doi:10.1523/JNEUROSCI.0399-12.2013.
54. Massa, S. M.; Xie, Y.; Yang, T.; Harrington, A. W.; Kim, M. L.; Yoon, S. O.; Kraemer, R.; Moore, L. A.; Hempstead, B. L.; Longo, F. M. Small, nonpeptide p75NTR ligands induce survival signaling and inhibit proNGF-induced death. J. Neurosci. 2006, 26, 5288-5300, doi:10.1523/JNEUROSCI.3547-05.2006.
55. Knowles, J. K.; Simmons, D. A.; Nguyen, T. V.; Vander Griend, L.; Xie, Y.; Zhang, H.; Yang, T.; Pollak, J.; Chang, T.; Arancio, O.; et al. Small molecule p75NTR ligand prevents cognitive deficits and neurite degeneration in an Alzheimer's mouse model. Neurobiol. Aging 2013, 34, 2052-2063, doi:10.1016/j.neurobiolaging.2013.02.015.
56. Coucha, M.; Shanab, A. Y.; Sayed, M.; Vazdarjanova, A.; El-Remessy, A. B. Modulating Expression of Thioredoxin Interacting Protein (TXNIP) Prevents Secondary Damage and Preserves Visual Function in a Mouse Model of Ischemia/Reperfusion. Int. J. Mol. Sci. 2019, 20, doi:10.3390/ijms20163969.
57. Herberg, S.; Kondrikova, G.; Hussein, K. A.; Periyasamy-Thandavan, S.; Johnson, M. H.; Elsalanty, M. E.; Shi, X.; Hamrick, M. W.; Isales, C. M.; Hill, W. D. Total body irradiation is permissive for mesenchymal stem cell-mediated new bone formation following local transplantation. Tissue Eng. Part A 2014, 20, 3212-3227, doi:10.1089/ten.TEA.2013.0663.
58. Mohamed, R.; Coucha, M.; Elshaer, S. L.; Artham, S.; Lemtalsi, T.; El-Remessy, A. B. Inducible overexpression of endothelial proNGF as a mouse model to study microvascular dysfunction. Biochim. Biophys. Acta 2018, 1864, 746-757, doi:10.1016/j.bbadis.2017.12.023.
59. Elshaer, S. L.; Abdelsaid, M. A.; Al-Azayzih, A.; Kumar, P.; Matragoon, S.; Nussbaum, J. J.; El-Remessy, A. B. Pronerve growth factor induces angiogenesis via activation of TrkA: Possible role in proliferative diabetic retinopathy. J. Diabetes Res. 2013, 2013, 432659, doi: 10.1155/2013/432659.
60. Park, H. S.; Ashour, D.; Elsharoud, A.; Chugh, R. M.; Ismail, N.; El Andaloussi, A.; Al-Hendy, A. Towards Cell free Therapy of Premature Ovarian Insufficiency: Human Bone Marrow Mesenchymal Stem Cells Secretome Enhances Angiogenesis in Human Ovarian Microvascular Endothelial Cells. HSOA J. Stem Cells Res. Dev. Ther. 2019, 5, doi:10.24966/srdt-2060/100019.
61. Carpentier, G.; Berndt, S.; Ferratge, S.; Rasband, W.; Cuendet, M.; Uzan, G.; Albanese, P. Angiogenesis Analyzer for ImageJ—A comparative morphometric analysis of “Endothelial Tube Formation Assay” and “Fibrin Bead Assay”. Sci. Rep. 2020, 10, 11568, doi:10.1038/s41598-020-67289-8.

We claim:

1. A method of treating ocular diseases that are associated with retinal ischemic injury in a subject comprising administering to the subject in need thereof a p75^{NTR} modulator.

2. The method of claim 1, wherein the p75^{NTR} modulator is LM11A-31, LM11A-24, THX-B, EVT901 or a derivative thereof.

3. The method of any one of claims 1-2, wherein ischemic areas on a retinal angiogram are reduced.

4. The method of any one of claims 1-3, further comprising administering to the subject a standard treatment for retinal ischemia.

5. The method of claim 4, wherein the standard treatment for retinal ischemia is anti-vascular endothelial grown factor (anti-VEGF) or steroids.

6. The method of any one of claims 1-5, wherein the p75^{NTR} modulator is administered orally.

7. The method of any one of claims 1-6, wherein the subject is not diabetic.

8. A method of improving visual acuity in a subject having a retinal ischemic injury comprising administering to the subject in need thereof a p75^{NTR} modulator.

9. The method of claim 8, wherein the p75^{NTR} modulator is LM11A-31, LM11A-24, THX-B, EVT901 or a derivative thereof.

10. The method of any one of claims 8-9, wherein ischemic areas on a retinal angiogram are reduced.

11. The method of any one of claims 8-10, further comprising administering to the subject a standard treatment for retinal ischemia.

12. The method of claim 10, wherein the standard treatment for retinal ischemia is anti-vascular endothelial grown factor (anti-VEGF) or steroids.

13. The method of any one of claims 8-12, wherein the p75^{NTR} modulator is administered orally.

14. The method of any one of claims 8-13, wherein the subject is not diabetic.

15. A method of preventing ischemia-induced retinal capillary degeneration in a subject comprising administering to the subject in need thereof a p75^{NTR} modulator, wherein the p75^{NTR} modulator inhibits the expression or activity of p75^{NTR} in mesenchymal stem cells (MSCs) in the retina of the subject.

16. The method of claim 15, wherein the p75^{NTR} modulator is LM11A-31, LM11A-24, THX-B, EVT901 or a derivative thereof.

17. The method of any one of claims 15-16, wherein ischemic areas on a retinal angiogram are reduced.

18. The method of any one of claims 15-17, wherein the p75^{NTR} modulator is administered orally.

19. A method of increasing vascular protection in an ischemic retina of a subject comprising administering to the retina of the subject in need thereof a p75^{NTR} modulator, wherein the p75^{NTR} modulator inhibits expression or activity of p75^{NTR} in mesenchymal stem cells (MSCs) in the retina of the subject.

20. The method of claim 19, wherein the p75^{NTR} modulator is LM11A-31, LM11A-24, THX-B, EVT901 or a derivative thereof.

21. The method of any one of claims 19-20, wherein ischemic areas on a retinal angiogram are reduced.

22. The method of any one of claims 19-21, wherein the p75^{NTR} modulator reduces p75^{NTR} mRNA expression.

23. The method of any one of claims 19-22, wherein the p75^{NTR} modulator is administered via intravitreal injection.

24. A method of increasing vascular homing of mesenchymal stem cells (MSCs) at a site of ischemic injury in a subject comprising administering to the subject in need thereof a p75^{NTR} modulator, wherein the p75^{NTR} modulator inhibits expression or activity of p75^{NTR} in the MSCs.

25. The method of claim 24, wherein the site of ischemic injury is in the retina of the subject.

26. The method of any one of claims 24-25, wherein the concentration of MSCs are increased in the retinal capillaries at the site of the ischemic injury in the subject's retina.

27. The method of any one of claims 24-26, wherein the p75^{NTR} modulator is LM11A-31, LM11A-24, THX-B, EVT901 or a derivative thereof.

28. The method of any one of claims 24-27, wherein the p75^{NTR} modulator is administered orally.

29. A method of improving mesenchymal stem cells (MSCs) secretome comprising inhibiting the expression or activity of p75^{NTR} in the MSCs.

30. The method of claim 28, wherein inhibiting the expression or activity of p75^{NTR} in MSCs comprises contacting the MSCs with a p75^{NTR} modulator.

31. The method of any one of claims 28-30, wherein the p75^{NTR} modulator is LM11A-31.

32. The method of claim 28, wherein inhibiting the expression or activity of p75^{NTR} in MSCs comprises genetically modifying the MSCs to decrease expression or activity of p75^{NTR} in the MSCs.

33. The method of claim 32, wherein genetically modifying the MSCs to have decreased expression or activity of p75^{NTR} comprises a reduction or a knock down mutation of p75^{NTR} in the MSCs.

34. The method of any one of claims 28-33, wherein expression of one or more of VEGF, SDF-1α, and NGF in the MSC is increased.

35. The method of any one of claims 28-34, wherein the MSCs are in culture.

36. The method of any one of claims 28-34, wherein the MSCs are in a retina of a subject.

37. A method of increasing survival factors in a human retinal endothelial cell (HRE) comprising contacting the HRE with a MSC having decreased p75^{NTR} expression or activity, wherein levels of VEGF-A, Akt, and Bcl-2 in the HRE is increased.

38. The method of claim 37, wherein contacting the HRE with the MSC having decreased p75^{NTR} expression or activity comprises first contacting the MSC with a p75^{NTR} modulator.

39. The method of claim 38, wherein the p75^{NTR} modulator is LM11A-31, LM11A-24, THX-B, EVT901 or a derivative thereof.

40. The method of claim 38, wherein contacting the HRE with the MSC having decreased p75^{NTR} expression or activity comprises contacting the HRE with a MSC comprising a reduction or a knock down mutation of p75^{NTR} in the MSCs.

41. A method of increasing angiogenic effects of a human retinal endothelial cell (HRE) comprising contacting the HRE with a MSC having decreased p75^{NTR} expression or activity, wherein HRE migration is increased.

42. The method of claim 41, wherein the ability of the HRE to form tubes is increased.

43. The method of claim 41, wherein HRE comprises a tube and wherein the tube length is increased in the HRE.

44. The method of any one of claims 41-43, wherein contacting the HRE with the MSC having decreased p75^{NTR} expression or activity comprises first contacting the MSC with a p75^{NTR} modulator.

45. The method of claim 44, wherein the p75^{NTR} modulator is LM11A-31, LM11A-24, THX-B, EVT901 or a derivative thereof.

46. The method of any one of claims 41-45, wherein contacting the HRE with the MSC having decreased p75^{NTR} expression or activity comprises contacting the HRE with a MSC a deletion or knocking out mutation of p75^{NTR} in the MSCs.

47. A method of decreasing urinary albumin excretion rate in a subject having diabetes comprising administering to the subject in need thereof a p75^{NTR} modulator.

48. A method of decreasing inflammation in the kidney of a subject having diabetes comprising administering to the subject in need thereof a p75^{NTR} modulator.

49. The method of claim 48, wherein the presence of IL-1β is decreased in the kidney or urine of the subject.

50. The method of claim 48, wherein the presence of IL-6 is decreased in the kidney of the subject.

51. The method of claim 48, wherein the presence of TNF-a is decreased in the kidney of the subject.

52. The method of claim 48, wherein the presence of NGF is increased in the kidney of the subject.

53. A method of decreasing IL-1β in a subject having diabetes comprising administering to the subject in need thereof a p75^{NTR} modulator, wherein IL-1β is decreased in one or both kidneys after administration of the p75^{NTR} modulator.

54. A method of decreasing IL-6 in a subject having diabetes comprising administering to the subject in need thereof a p75^{NTR} modulator, wherein IL-6 is decreased in one or both kidneys after administration of the p75^{NTR} modulator.

55. A method of decreasing TGF-β in a subject having diabetes comprising administering to the subject in need thereof a p75^{NTR} modulator, wherein TGF-β is decreased in one or both kidneys after administration of the p75^{NTR} modulator.

56. A method of decreasing one or more markers of fibrosis in a subject having diabetes comprising administering to the subject in need thereof a p75^{NTR} modulator, wherein one or more markers of fibrosis is decreased in one or both kidneys after administration of the p75^{NTR} modulator.

57. The method of claim 56, wherein the markers of fibrosis are one or more of fibronectin and α-SMA.

58. A method of decreasing fibrotic tissue in a kidney of a subject having diabetes comprising administering to the subject in need thereof a p75^{NTR} modulator.

59. The method of any one of claims 47-58, wherein the subject having diabetes has advanced stage diabetes.

60. A method of treating late stage diabetes in a subject comprising administering to the subject in need thereof a p75^{NTR} modulator.

61. The method of any one of claims 47-60, wherein the p75^{NTR} modulator is LM11A-31, LM11A-24, THX-B, EVT901 or a derivative thereof.

62. A method of treating an ocular disease associated with retinal ischemic injury in a subject comprising administering to the subject in need thereof one or more mesenchymal stem cells (MSCs) comprising decreased p75^{NTR} expression or activity.

63. A method of treating retinal ischemic injury in a subject comprising administering to the subject in need thereof one or more MSCs comprising decreased p75^{NTR} expression or activity.

64. A method of improving visual acuity in a subject having a retinal ischemic injury comprising administering to the subject in need thereof one or more MSCs comprising decreased p75NTR expression or activity.

65. A method of preventing ischemia-induced retinal capillary degeneration in a subject comprising administering to the subject one or more MSCs comprising decreased p75NTR expression or activity.

66. A method of increasing vascular protection in an ischemic retina of a subject comprising administering to the retina of the subject one or more MSCs comprising decreased p75NTR expression or activity.

67. A method of increasing vascular homing of MSCs at a site of ischemic injury in a subject comprising administering to the subject one or more MSCs comprising decreased p75NTR expression or activity.

68. The methods of any of claims 62-67, wherein prior to administering the subject the one or more MSCs comprising decreased p75NTR expression or activity, the MSCs are treated with a p75NTR modulator.

69. The method of claim 68, wherein the p75NTR modulator causes a decrease in p75NTR activity or expression in the MSCs.

70. A method of treating an ocular disease associated with retinal ischemic injury in a subject comprising administering to the subject in need thereof a MSC secretome collected from one or more MSCs comprising decreased p75NTR expression or activity.

71. A method of treating retinal ischemic injury in a subject comprising administering to the subject in need thereof a MSC secretome collected from one or more MSCs comprising decreased p75NTR expression or activity.

72. A method of preventing ischemia-induced retinal capillary degeneration in a subject comprising administering to the subject a MSC secretome collected from one or more MSCs comprising decreased p75NTR expression or activity.

73. A method of increasing vascular protection in an ischemic retina of a subject comprising administering to the retina of the subject a MSC secretome collected from one or more MSCs comprising decreased p75NTR expression or activity.

74. A method of increasing vascular homing of MSCs at a site of ischemic injury in a subject comprising administering to the subject a MSC secretome collected from one or more MSCs comprising decreased p75NTR expression or activity.

75. A method of decreasing one or more markers of fibrosis in a non-diabetic subject comprising administering to the subject in need thereof a p75NTR modulator, wherein one or more markers of fibrosis is decreased in one or both kidneys after administration of the p75NTR modulator.

76. The method of claim 75, wherein the markers of fibrosis are one or more of fibronectin and α-SMA.


SEQUENCE LISTING (17 sequences)

SEQ ID NO: 1 - RNA, 63 nt, synthetic construct
  source 1..63: mol_type = other RNA
gatccgaggatcggaggcttgtcattcaagagatgacaagcctccgatcctcttttttggaaa

SEQ ID NO: 2 - DNA, 20 bp, synthetic construct
  source 1..20: mol_type = other DNA
cctgcctggacagtgttacg

SEQ ID NO: 3 - DNA, 20 bp, synthetic construct
  source 1..20: mol_type = other DNA
cacacagggagcggacatac

SEQ ID NO: 4 - DNA, 20 bp, synthetic construct
  source 1..20: mol_type = other DNA
gtacctccaccatgccaagt

SEQ ID NO: 5 - DNA, 20 bp, synthetic construct
  source 1..20: mol_type = other DNA
gcattcacatctgctgtgct

SEQ ID NO: 6 - DNA, 21 bp, synthetic construct
  source 1..21: mol_type = other DNA
atgaacgacgtagccattgtg

SEQ ID NO: 7 - DNA, 22 bp, synthetic construct
  source 1..22: mol_type = other DNA
ttgtagccaataaaggtgccat

SEQ ID NO: 8 - DNA, 19 bp, synthetic construct
  source 1..19: mol_type = other DNA
gtggtggaggaactcttca

SEQ ID NO: 9 - DNA, 20 bp, synthetic construct
  source 1..20: mol_type = other DNA
gttccacaaaggcatcccag

SEQ ID NO: 10 - DNA, 20 bp, synthetic construct
  source 1..20: mol_type = other DNA
agcaaactggtgctcaaggc

SEQ ID NO: 11 - DNA, 20 bp, synthetic construct
  source 1..20: mol_type = other DNA
ccacaaagatggtcactgtc

SEQ ID NO: 12 - DNA, 20 bp, synthetic construct
  source 1..20: mol_type = other DNA
cctcagagagacattcatgg

SEQ ID NO: 13 - DNA, 20 bp, synthetic construct
  source 1..20: mol_type = other DNA
gcagtagtcgcctctgaaga

SEQ ID NO: 14 - DNA, 24 bp, synthetic construct
  source 1..24: mol_type = other DNA
ggaaatttgtatcccgagtatctg

SEQ ID NO: 15 - DNA, 23 bp, synthetic construct
  source 1..23: mol_type = other DNA
gtcttccagtgtgatgatggtaa

SEQ ID NO: 16 - DNA, 20 bp, synthetic construct
  source 1..20: mol_type = other DNA
cacattgggggtaggaacac

SEQ ID NO: 17 - DNA, 21 bp, synthetic construct
  source 1..21: mol_type = other DNA
aactttgggcattgtggaagg